(19) United States
(12) Patent Application Publication
(10) Pub. No.: US 2024/0081633 A1
(43) Pub. Date: Mar. 14, 2024

(54) METHODS AND APPARATUS FOR RECONFIGURABLE OPTICAL ENDOSCOPIC CATHETER

(71) Applicant: Leadoptik Inc., San Jose, CA (US)

(72) Inventor: Mohammadreza Khorasaninejad, Milpitas, CA (US)

(21) Appl. No.: 18/508,852

(22) Filed: Nov. 14, 2023

Related U.S. Application Data
(63) Continuation-in-part of application No. PCT/US2022/029301, filed on May 13, 2022.
(60) Provisional application No. 63/189,053, filed on May 14, 2021.

(51) Int. Cl. A61B 1/07; A61B 1/06; G02B 5/00; G02B 5/18

(52) U.S. Cl. CPC A61B 1/07; A61B 1/0615; A61B 1/0638; A61B 1/0655; G02B 5/001; G02B 5/1814; A61B 5/0066

(57) ABSTRACT

Several configurations of optical systems are disclosed herein. In some embodiments, the optical system includes a substrate having a first surface and a second surface, and a first reflector disposed on the substrate and configured to receive light. The light includes at least one of a first wavelength of light and a second wavelength of light. The first reflector is configured to reflect the first wavelength of light along a first light path toward a first diffractive lens and to transmit the second wavelength of light toward a second reflector. The second reflector is configured to reflect the second wavelength of light along a second light path toward a second diffractive lens.

### CROSS-REFERENCE TO RELATED APPLICATION(S)

This application is a continuation-in-part of International Application No. PCT/US2022/029301, “Methods And Apparatus For Reconfigurable Optical Endoscopic Catheter,” filed May 13, 2022; which claims priority to U.S. Provisional Patent Application Ser. No. 63/189,053, “Methods and Apparatus for Reconfigurable Optical Endoscopic Catheter,” filed May 14, 2021. The subject matter of all of the foregoing is incorporated herein by reference in its entirety.

### TECHNICAL FIELD

The present disclosure relates generally to miniaturized optical imaging and illuminating systems, apparatus, and devices. More specifically, this disclosure presents methods, systems, apparatuses, and devices to realize miniaturized medical imaging based on the optical endoscopic catheter including the Optical Coherence Tomography, Raman Spectroscopy, and/or Fluorescence Spectroscopy techniques.

### BACKGROUND

Accurate diagnosis and treatment of diseases in luminal organs such as the coronary arteries, the pulmonary airways, and the gastrointestinal tract are difficult due to the inaccessibility of lesions, particularly in in-vivo cases. This is the main drive behind the miniaturization of optical imaging and illuminating (for therapeutic purposes) systems. One of the commonly used imaging systems is the endoscopic optical coherence tomography (OCT) catheter. In a typical endoscopic catheter, optical power is delivered via an optical fiber to the distal end of the catheter and then it is re-directed and focused into the tissue via several cascaded optical components. Two common approaches of re-directing and focusing the light are based on (i) graded-index (GRIN) lenses and prisms and (ii) angle polished ball lenses. In the former, the GRIN lens focuses the light and then the prism re-directs the light toward the tissue (in the radial direction, relative to the length of fiber) where one needs to perform imaging and/or light illumination. The latter can be seen as a prism and a lens integrated into one device, where the angle polished facet section re-directs (often by 90 degrees) the light coming from the fiber toward the lens, and the lens focuses the light into a tissue. In the case of imaging, scattered light from the tissue is collected by the same lens and re-directed toward the fiber via angle polished facet. Then the fiber delivers this light to a post-processing system (often to an interferometric arm and detectors) for processing and forming images. The endoscopic catheter (including fiber and other optical components attached to it) is moved back and forth and rotated along its axis (e.g., about a longitudinal axis which runs the length of the fiber) to reconstruct a 3D image of the scene (e.g., tissue).

### SUMMARY OF THE DISCLOSURE

This summary is provided to introduce a selection of embodiments in a simplified form. Embodiments will be described in further detail below in the Detailed Description. This summary is not intended to identify key features or essential features of the claimed subject matter, nor is this summary intended to be used to limit the claimed subject matter's scope.

According to some embodiments, an apparatus for dynamically controlling propagation directions and shape of light (e.g., focusing, expanding/condensing the beam width, coupling in and out of substrate), sorting light based on its properties (e.g., polarization, angle, and/or wavelength) is disclosed.

In an aspect, an optical system is disclosed. The optical system includes a substrate having a first surface and a second surface, and a first reflector disposed on the substrate and configured to receive light. The light includes at least one of a first wavelength of light and a second wavelength of light. The first reflector is configured to reflect the first wavelength of light along a first light path toward a first diffractive lens and to transmit the second wavelength of light toward a second reflector. The second reflector is configured to reflect the second wavelength of light along a second light path toward a second diffractive lens.

In another aspect, an optical system is disclosed having a substrate with a first surface and a second surface, and a collimator configured to receive and collimate input light. The input light includes at least one of a first wavelength of light and a second wavelength of light. A first reflector is configured to reflect the first wavelength of light toward a first diffractive lens and to transmit the second wavelength of light toward a second reflector. The second reflector is configured to reflect the second wavelength of light toward a second diffractive lens.

The majority of embodiments include a light source, optical fiber, diffractive optical components (e.g., diffractive lens, diffractive gratings, metasurface-based lenses, metasurface-based grating), refractive optical elements (e.g., mirror, wavelength selective mirror, partial mirror, substrate) and/or liquid crystals (LC), thin films, and polarization films (e.g., polarization reflector, absorptive polarizer, half waveplate, quarter waveplate) to control, shape, sort, and guide light toward a desired direction and ultimately focus it into an object for imaging and/or illumination. Further, embodiments may include at least one optical source, at least one sensor, and at least one control module. The control module may control, tune, and adjust the functionality of each component depending on feedback from the sensor or from a user. The functionality of some components can be dynamically changed by applying an electric voltage and/or current or by changing the properties of impinging light (e.g., polarization, wavelength, angle). Further, the polarization state of light may be linear, circular, elliptical, random, unpolarized, or any arbitrary combination of them.

The methods disclosed here may include a step of receiving, using a communication device or sensor, feedback data from at least one sensor, or image processing software. Using this feedback, the control module may adjust the functionality of one or more components, and/or change the wavelength, polarization, or other properties of the input light to improve or adjust the performance of the system/device for imaging and/or illuminating purposes.

Both the foregoing summary and the following Detailed Description provide examples and are explanatory only. Accordingly, the foregoing summary and the following Detailed Description should not be considered to be restrictive. In addition to those set forth herein, further features or variations may be provided. For example, embodiments may be directed to various feature combinations and sub-combinations described in the Detailed Description.

### BRIEF DESCRIPTION OF THE DRAWINGS

The accompanying drawings presented in this disclosure partially constitute the disclosure and illustrate different embodiments. The incorporated drawings may contain representations of various copyrights and trademarks owned by the Applicant. All rights to various trademarks and copyrights represented herein are vested in and the property of the Applicant. The Applicant retains and reserves all rights in their trademarks and copyrights included herein, and grants permission to reproduce the material only in connection with reproduction of the granted patent and for no other purposes.

Furthermore, the drawings may contain captions and/or text that may explain certain embodiments of the present disclosure. These text and captions are included for non-limiting, explanatory, and illustrative purposes of certain embodiments described in the present disclosure.

FIG. 1A-D show an embodiment of small form-factor endoscopic fiber-based imaging and illuminating system.

FIG. 2A-B show multi-spectral and multi-zoom imaging embodiments using cascaded wavelength-selective reflectors.

FIG. 3A-B show multi-spectral and multi-zoom imaging and illuminating embodiments that utilize the dispersive response of diffractive gratings.

FIG. 4A-E show five embodiments of miniaturized polarization-resolved imaging and illuminating system.

FIG. 5A-D show multifunctional optical imaging and illuminating embodiments.

FIG. 6A-C show three embodiments configured to extend the depth of focus of Optical Imaging and Illuminating Systems (“OIIS”)

FIG. 7A-C show embodiments having Optical Imaging and Illuminating Systems with a reconfigurable focal length.

FIG. 8A-B show an exploded view and cross-section views of an embodiment of an optical imaging and illuminating system illustrating different integration schemes with various components.

FIG. 9 shows a block diagram of different modules for implementing the technologies disclosed herein.

### DETAILED DESCRIPTION

Embodiments of the present invention are described more fully hereinafter with reference to the accompanying drawings; however, alternative configurations and embodiments are also possible without departing from the scope of the present application. Thus, the present application should not be construed as limited to the embodiments set forth herein. Rather, the illustrated and described embodiments are provided as examples to convey the scope of the invention to those skilled in the art. In the drawings, the size and relative sizes of layers and regions may be exaggerated for clarity.

It will be understood that, although the terms first, second, third, etc. may be used herein to describe various elements, components, regions, layers, and/or sections, these elements, components, regions, layers, and/or sections should not be limited by these terms. These terms are used to distinguish one element, component, region, layer, or section from another region, layer, or a section. Thus, a first element, component, region, layer, or section discussed below could be termed a second element, component, region, layer, or section without departing from the teachings of the present disclosure.

As used herein, the singular forms “a,” “an,” and “the” are intended to include the plural forms as well, unless the context clearly indicates otherwise. It will be further understood that the terms “comprises” and/or “compromising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items and may be abbreviated as “/”.

Throughout this disclosure, the term “arbitrary” may be used herein to describe of being any material, shape, size, features, order, type or kind, orientation, position, quantity, components, and arrangements of components with single and/or combinations of components that may allow the present disclosure or that specific component to fulfill the objectives, function, and intents of the present invention or that specific component/system within the invention.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

By way of introduction, conventional optical illuminating and imaging systems as described previously have various drawbacks. For example, systems that rely on GRIN and ball lenses suffer from significant optical aberration including spherical aberration and astigmatism, which degrade the imaging resolution. Although one can mitigate these aberrations by cascading several lenses similar to microscope objective lenses, the large size and high cost of systems with multiple lenses render this approach expensive and impractical.

Besides the low imaging resolution, refractive-based lenses (e.g., spherical lenses) have limited functionality. They cannot perform polarization-resolved imaging or multispectral imaging, and their focal lengths are fixed (i.e., cannot be adjusted or changed). Furthermore, these lenses should be cascaded with other bulky optical components such as prisms to perform imaging in the radial direction (e.g., orthogonal to fiber length in fiber-based endoscope), which hinders further miniaturization of the imaging system. Because most of the fiber-based endoscope designs are based on the finite/finite conjugate design (point to point focusing and imaging, fiber core to focal spot, and vice versa) the optical path error (e.g., due to fabrication tolerances) between the fiber and lens not only can cause aberration and reduce the resolving power but also can change the effective focal length of the imaging system. Due to its solid nature, using a prism makes it very difficult to place any other component between the fiber and the lens, thereby limiting the functionality of the whole system. For example, it may be very hard to control or sort the light, which is transmitted between fiber and lens via a prism, based on its polarization and/or wavelength. Also, both refractive lenses and prisms are passive optical components without adjustability, which prevents the optical system from being tuned or dynamically operated. In this disclosure, several systems and methods are described that address these problems and shortcomings.

The present disclosure describes devices, apparatuses, and systems to facilitate light control for imaging and illuminating purposes in a compact and small form-factor. Further, the present disclosure describes various methods to enable multi-zoom imaging, multi-spectral imaging, and polarization-resolved imaging. Further, the present disclosure relates generally to multi-functional small form-factor optical systems to focus light into tissue/organs for imaging and illumination via an optical fiber and stack of miniaturized optical components and devices. Optical components and devices can be based on diffractive optics, metasurfaces, and refractive optics and/or combinations thereof.

In the present disclosure, diffractive components (e.g., gratings, lenses) include arbitrary arrays of subwavelength scatterer, resonator, and/or nanostructures. These scatterers, resonators, and/or nanostructures may be referred to herein as building blocks. Building blocks can individually or collectively control one or more basic properties of light such as phase, amplitude, polarization, spatial and temporal profile, the direction of propagation, angle of rays, or combinations of these properties at the same time. For example, diffractive lenses are very thin lenses that can focus, diverge or converge the impinging light. Incoming light can have arbitrary profile and/or angular distribution. In general, diffractive gratings diffract impinging light to one or several different orders (e.g., ±1, ±2, ±3, etc.) depending on the design parameters (e.g., pitch and/or pattern) of the gratings. Diffractive axicons can generate Bessel beams of different orders (e.g., Jo, Ji, etc.). Bessel beams have unique non-diffractive properties where light can stay focused for an extended distance compared to other counterparts such as diffractive lenses. Diffractive components' building blocks may be made of materials including semiconductors (e.g., amorphous silicon, polycrystalline silicon, silicon carbide, gallium nitride, gallium phosphide), crystals (e.g., silicon, lithium niobate, diamond), dielectrics (e.g., silicon dioxide, silicon nitride, aluminum oxide, hafnium oxide, titanium dioxide, indium oxide), polymers (e.g., photoresist, PMMA), metals (e.g., silver, aluminum, gold), two-dimensional (2D) materials (e.g., graphene, boron nitride), phase change materials (e.g., chalcogenide, vanadium dioxide) or any mixtures or alloys thereof.

In the present disclosure, metasurfaces are advanced forms of diffractive components and may be referred to as meta-gratings (gratings based on metasurface designs), meta-lenses (lenses based on metasurface designs), and meta-hologram (holograms based on metasurface designs). These metasurfaces are multifunctional flat components with engineered dispersive, polarization, and angular responses and may be fabricated using various approaches such as optical lithography, deep-ultraviolet lithography, electron beam lithography, nanoimprinting, reactive ion etching, electron beam deposition, sputtering, plasma-enhanced deposition, atomic layer deposition, and any combination of the aforementioned processes with any arbitrary orders. Metasurface building blocks may be made of similar materials mentioned above for diffractive components.

Throughout the present disclosure, “optical fiber,” which may also be referred to herein as just “fiber,” may refer to a flexible, transparent fiber made by drawing glass (silica) or plastic, or other materials. Optical fibers referred to herein may include single-mode fiber, multimode fiber, photonic crystal fiber, and any other special-purpose fiber. Fiber may be connected to a bare ferrule, or connector comprising ferrule. Ferrule type may be a ferrule connector (“FC”), Lucent connector, angle polished connector (“APC”), physical contact (“PC”) connector, Ultra-Physical Contact (UPC), or any combination of them. Other connectors may be used without departing from the scope of the present disclosure. The ferrule can be made of glass, ceramic, plastic, or any other materials. Fiber connectors can be FC, PC, APC, subscriber connector (“SC”), or any combination thereof. The ferrule may be customized with arbitrary shapes and sizes. The operating wavelength of fiber can be Ultra-Violet (UV), visible, Near Infrared (NIR), Short-Wave Infrared (SWIR), or/and longer or shorter wavelengths. Fibers can have a protective layer, or may be enclosed with other plastic tubes, polymer tubes, glass tubes, and/or torque coils. Different types of tubes (e.g., plastic, polymer, glass) generally are used as a protective enclosure for optical systems and devices. A torque coil is used to transfer torque to the optical system (e.g., the imaging/illuminating probe) for rotation and thus performs radial imaging/illuminating.

In the present disclosure, the term “optical source” refers to a coherent, partially coherent, or incoherent light source that may be based on any technology such as, but not restricted to, swept-source laser, light-emitting diodes (LEDs), edge-emitting semiconductor laser diodes, vertical-cavity surface-emitting lasers (VCSELs), supercontinuum sources, superluminescent diodes, white light sources, and halogen lamps. The wavelength of the light source may be in deep-UV, UV, visible, NIR, SWIR, mid-infrared, or far-infrared ranges depending on the application of the catheter (for example, for imaging, or therapeutic applications wavelength may be different). The light may be delivered as pulses of energy (e.g., pulse laser) or as a Continuous Wave (CW).

Throughout the present disclosure, the term “color filter” refers to a device that selectively transmits or reflects light of different colors (i.e., wavelengths). Color filters can be based on various mechanisms such as absorption (e.g., using a dye, pigment, plasmonic particles, metallic nanostructures), interference (e.g., thin-film, subwavelength grating, Mie resonance structure, plasmonic and metallic nanostructure), or diffraction (e.g., reflective or transmission grating). In this disclosure, a mirror may refer to a device that reflects incident light. The reflectivity of the mirror can be smaller or larger than 10%, smaller or larger than 25% smaller or larger than 75%, or smaller than 100%. The reflectivity of the mirror may be a function of light wavelength, polarization, and/or its angle of incidence.

Throughout the present disclosure, the imaging sensor may refer to any arbitrary imaging and sensing technologies to detect or capture light intensity or other light properties such as phase, angle, polarization, and wavelength. Some examples of such arbitrary imaging and sensing technologies include complementary-symmetry metal-oxide-semiconductor (CMOS), charge-coupled device (CCD), intensified charge-coupled device (ICCD), scientific CMOS (sCMOS), avalanche diode (AD), time-of-flight (ToF), Schottky diodes or any other light or electromagnetic sensing mechanism operating at deep-UV, visible, SWIR, NIR, far-infrared and/or other wavelengths.

Further, the present disclosure describes a hybrid approach based on refractive optics, diffractive optics, metasurface, and other flat optical technology (e.g., polarizers, waveplates, quarter waveplates, half-wave plates, mirrors, reflectors, partial reflectors, and color filters). The dynamic capability of various optical systems described herein may be enabled by including components configured to achieve electro-optic (e.g., by injecting carrier) or thermo-optic (e.g., by local heating) effects. Other mechanisms and devices such as LC may also be used to provide adjustability within optical systems. The dynamic capability may significantly enhance the performance and flexibility of optical systems. The multifunctional nature of cascaded planar components enables such dynamic systems to satisfy small form-factors necessary for in-vivo medical applications. The main focus of the present disclosure is on enabling small form-factor, reconfigurable, high-performance optical systems for medical imaging, diagnostic, and therapeutic purposes.

Throughout the present disclosure, dynamic components or design or in general the adjective “dynamic” as used herein may refer to components or designs having function, performance, and properties that can be adjusted over time by selectively changing the properties of light (e.g., polarization, wavelength, intensity) in response to one or more of an external optical, thermal, electrical, or mechanical signal.

Simulations in this disclosure are performed using ray-tracing methods considering the law of reflection, refraction, and diffraction. For all simulations described and illustrated herein, each ray is assumed to have a single wavelength with a very small bandwidth for sake of simplicity. It is important to note that in experiments and in a real device, rays (e.g., input light) may have considerable bandwidth which may be smaller or larger than 10 nm, smaller larger than 25 nm, smaller or larger than 50 nm, smaller or larger than 100 nm. In some embodiments, the bandwidth is between approximately 50 nm and approximately 100 nm.

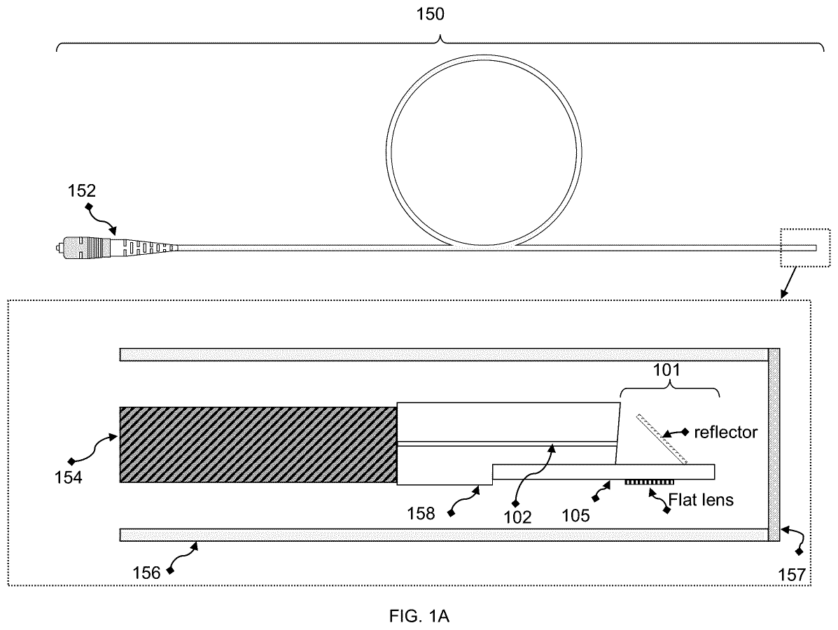

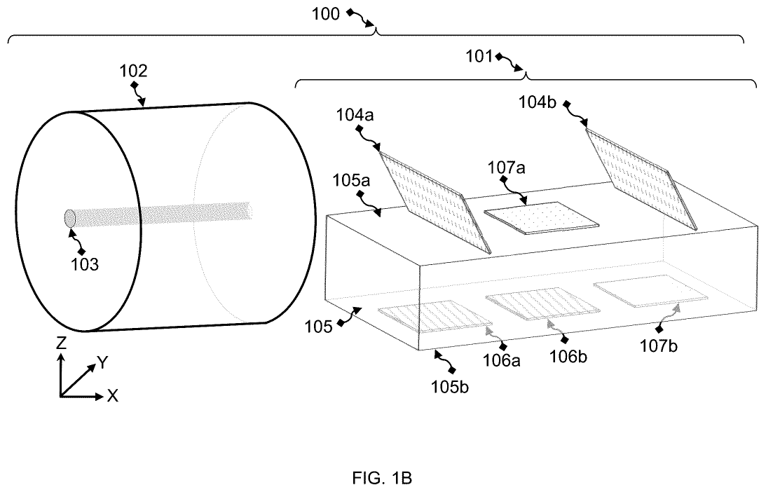

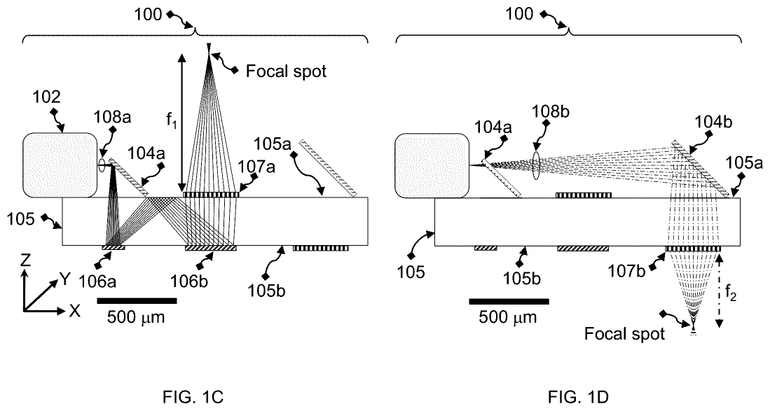

FIG. 1A (top) shows a schematic of an endoscopic catheter 150 including a fiber connector 152, an optical fiber 102, a torque coil 154 (to transfer torque from one end of the catheter to the other), and other optical and mechanical components enclosed by a sheath 156. The magnified view at the bottom of FIG. 1A shows the components at the distal end of the endoscopic catheter including the torque coil 154 which is connected to an optical imaging and illuminating system (“OIIS”) 101 via a ferrule 158. Ferrule 158 holds the end of the fiber 102. Fiber 102 passes through the torque coil 154 and connects to the fiber connector 152 at the other end of endoscopic catheter 150 (See FIG. 1A top). Torque coil 154, ferrule 158 and OIIS 101 are enclosed by a sheath 156. Sheath 156 may be a transparent plastic, polymer or glass tube or combination of them. The end of sheath 156 may be sealed by an enclosure cap 157 (e.g., plastic or glass substrate, silicone gel, etc.).

FIG. 1B shows a perspective view of an embodiment of one end of a small form-factor endoscopic catheter 100. In this embodiment, fiber 102 includes a core 103 configured to receive light from an optical source (not shown). In some embodiments, fiber 102 may be connected to a ferrule (see FIG. 1A). Fiber 102 delivers the light to OIIS 101. In some embodiments, the OIIS may have dimensions in the range of approximately 0.2-1.5 mm in the z-dimension, approximately 0.2-1.5 mm in the y-dimension, and approximately 1-5 mm in the x-dimension. Various embodiments may have a cross-section of not more than 0.5 mm×0.5 mm, a cross-section of not more than 1.5 mm×1.5 mm, or a cross-section of not more than 2 mm×2 mm. Various embodiments may have a length of not more than 5 mm, or a length of not more than 10 mm. Size constraints in the x-dimension may be less restrictive than those in the y- and z-dimensions for certain applications.

In the endoscopic catheter 100, the OIIS 101 comprises two wavelength-selective reflectors (“WSR”) 104a and 104b, two diffractive gratings 106a and 106b, and two diffractive lenses 107a and 107b disposed on or in or otherwise supported by a substrate 105. Substrate 105 may be made of materials including glass (e.g., fused silica, Pyrex, high index glass, quartz), semiconductors (e.g., amorphous silicon, polycrystalline silicon, silicon carbide, gallium nitride, gallium phosphide), crystals (e.g., sapphire silicon, lithium niobate, diamond), dielectrics (e.g., silicon dioxide, silicon nitride, aluminum oxide, hafnium oxide, titanium dioxide, indium oxide), polymers (e.g., photoresist, PMMA). Here, for exemplary purposes, a glass substrate is considered. One or more of diffractive lenses 107a, 107b may be replaced with Fresnel lenses, metasurface-based lenses, and/or refractive lenses (e.g., spherical lenses, aspherical lenses, free-form lenses). The WSRs 104a and 104b may be disposed on a first surface 105a. The WSRs may be positioned at an angle (e.g., approximately 37 degrees, 45 degrees, or 50 degrees) relative to the first surface 105a. The reflection value of WSR for the desired wavelength may be smaller or larger than 95%, smaller or larger than 90%, and smaller or larger than 80%, smaller or larger than 70% while allowing other wavelengths to pass through with a maximum transmission value smaller or larger than 95%, smaller or larger than 90%, smaller or larger than 80%, smaller or larger than 70%. In some embodiments, the reflection value of WSR for the desired wavelength may be between approximately 80% and approximately 95%. In some embodiments, the transmission value of WSR for the desired wavelength may be between approximately 80% and approximately 95%. The first diffractive lens 107a may also be disposed on the first surface 105a in between the two WSRs 104a and 104b. The two diffractive gratings 106a and 106b as well as the second diffractive lens 107b may be disposed on a second surface 105b opposite the first surface 105a of the substrate. The first and second surfaces 105a and 105b may be substantially parallel or may have an angle relative to each other. This angle can be smaller or larger than 5 degrees, smaller or larger than 10 degrees. The first and second surfaces 105a and 105b may be planar and substantially parallel to each other. The positions, sizes, and shapes of each component on the substrate 105 may be selected to receive and direct light in a specific way, as will be discussed herein below with respect to FIGS. 1B and 1C.

For applications where the endoscopic catheter 100 is used for optical coherence tomography, the operating wavelength (i.e., the wavelength of light received by the OIIS) may be in the NIR or SWIR regions (e.g., wavelengths between 800 nm to 1700 nm). Such wavelengths advantageously allow for the penetration of light into the tissue for depth imaging and illumination. For systems using wavelengths in the range of approximately 800 nm to approximately 1700 nm, diffractive lenses can include an array of silicon nanostructures on a glass substrate. Silicon has a high refractive index (e.g., refractive index n>3) and negligible material loss in this wavelength range. Thus, silicon nanostructures on a glass substrate may enable the low-loss and strong light-nanostructures interaction necessary to make high-efficiency and high-performance flat devices and components.

FIGS. 1C and 1D show side views of the endoscopic catheter 100 working in different ways, where the method of operation is a function of input light wavelength. Operation of the endoscopic catheter 100 with input light having a wavelength of approximately 1300 nm (i.e., ray 108a) is shown in FIG. 1C while the operation of the endoscopic catheter 100 with input light having a wavelength of approximately 800 nm (i.e., ray 108b) is shown in FIG. 1D. Light rays 108a and 108b may be delivered to the endoscopic catheter 100 simultaneously, but the ray trace simulations are divided into two figures for simplicity. While first and second wavelengths of 1300 nm and 800 nm are used as an example, other wavelengths of light may be selected without departing from the scope of the present disclosure.

In the ray-tracing simulation shown in FIG. 1C, rays 108a with the wavelength of 1300 nm exit the fiber facet 102 traveling toward and facing WSR 104a. WSR 104a is angled at approximately 45 degrees relative to the direction of travel of the rays 108a. The WSR 104a is designed to reflect the light rays 108a centered at 1300 nm wavelength and to allow light rays 108b centered at 800 nm wavelength to pass through undisturbed (as shown in FIG. 1D). Thus, the rays 108a impinge upon and are reflected by the first WSR 104a towards substrate 105. The operation bandwidth of WSRs (104a, 104b) can be adjusted depending on design parameters. For example, the operation bandwidth can be smaller or larger than 10 nm, smaller or larger than 25 nm, smaller or larger than 50 nm, or smaller or larger than 100 nm. In some embodiments, the bandwidth may be between approximately 50 nm and approximately 100 nm.

Rays 108a reflected by WSR 104a enter substrate 105 substantially perpendicular to the first surface 105a. The rays 108a travel through the substrate 105 toward the second surface 105b on which is disposed the diffractive grating 106a. The diffractive grating 106a is sized and positioned to intercept the rays 108a, accounting for a small amount of light divergence that may occur. The diffractive grating 106a diffracts the rays 108a into angles larger than the Total Internal Reflection (TIR) angle of the substrate. Diffracted light bounces off of the first surface 105a due to TIR and travels toward second diffractive grating 106b disposed on the second surface 105b. The second diffractive grating 106b is designed to diffract light such that, after the diffraction event, the light travels through the substrate 105 at an angle substantially perpendicular to the first and second surfaces 105a and 105b. A diffractive lens 107a is positioned on the first surface 105a such that it receives light diffracted from the second diffractive grating 106b. Diffractive lens 107a may be sized to account for an increase in divergence of the light rays 108a as they traveled through the optical system. The diffractive lens 107a focuses rays 108a into a diffraction-limited spot at a focal length (e.g., f₁=0.9 mm) relative to the first surface 105a of the substrate 105. Here, the focal length of 0.9 mm is selected for exemplary purposes and the focal length may be smaller or larger than 1 mm, smaller or larger than 5 mm, smaller or larger than 10 mm without departing from the scope of the present disclosure.

Referring to FIG. 1D, a second method of operation of the endoscopic catheter 100 is shown with input light rays 108b having wavelength of 800 nm. As discussed above, the light rays 108b pass through WSR 104a unperturbed until they encounter the second WSR 104b. The second WSR 104b is designed to reflect light centered at 800 nm; thus, rays 108b are reflected by the second WSR 104b toward the substrate. The rays 108b travel through the substrate 105 toward the second diffractive lens 107b which is positioned on the second surface 105b and is configured to receive the reflected rays 108b. Rays 108b will be focused by the diffractive lens 107b at a second focal length (f₂=0.5 mm) relative to the second surface 105b of the substrate 105. Thus, by utilizing reflectors (WSRs 104a and 104b) that only reflect certain bandwidths of light, an OIIS with adjustable focal length dependent on input light wavelength is demonstrated. Here the endoscopic catheter 100 may be enclosed by a protective tube/sheath made of glass, polymer, or plastic. In this case, the diffractive lenses 107a and 107b may be designed to account for optical paths added by the protective tube/sheath.

Notably, diffractive lenses may have chromatic aberration whereby changing the wavelength of input light causes the focal spot size to become larger than the diffraction limit, and focusing efficiency degrades. However, in the catheter 100, each of the diffractive lenses 107a and 107b can be designed for a particular operating wavelength (e.g., 1300 nm and 800 nm, respectively). This enables each diffractive lens to achieve optimal performance in terms of imaging resolution and focusing efficiency. One other important point about endoscopic catheter 100 is that the light exiting the fiber facet 102 is diverging. By controlling the optical path length that light travels before reaching the diffractive lens, the beam waist may also be controlled. The longer the light travels, the larger the beam waist becomes. Therefore, for a fixed focal length (or working distance of the lenses) the numerical aperture (“NA”) of the OIIS may also be controlled, assuming the beam waist is equal to the diameter of the lens used to focus the light. Another advantage of having two focal spots (on top and bottom of OIIS) is to increase the imaging speed and/or frame rate. Generally, OIIS rotates along the fiber axis (X-direction) to perform 3D imaging. Having focal spots on top and bottom, one may perform full radial imaging by rotating the OIIS 180 degree (rather than 360 degrees). In other words, top lens 107a forms an image of the top half-circle, and bottom lens 107b forms the bottom half-circle. By stitching these two images using, for example, image post-processing software, one may reconstruct the full image by only rotating OIIS 101 through 180 degrees which can enhance the speed of imaging. In another scenario where OIIS 101 rotates 360 (degrees), the frame rate may be doubled by combing images captured by the top and bottom lenses 107a and 107b.

In addition to using the OIIS 101 to image a surrounding environment as discussed above, the same OIIS 101 may be utilized for illumination of the surrounding environment (e.g., therapeutic purposes). A therapeutic protocol may require the use of multiple different wavelengths of light, for example, light having wavelengths in the UV or visible wavelength range. Depending on the particular wavelengths to be used, other materials, such as titanium dioxide or hafnium dioxide (with negligible absorption loss in these wavelength ranges and relatively high refractive index of n=˜2.5), may be used to form one or more of the components, such as the diffractive gratings or diffractive lenses. The titanium dioxide or hafnium dioxide components may be better suited to assembly on a different type of substrate depending on operating wavelength.

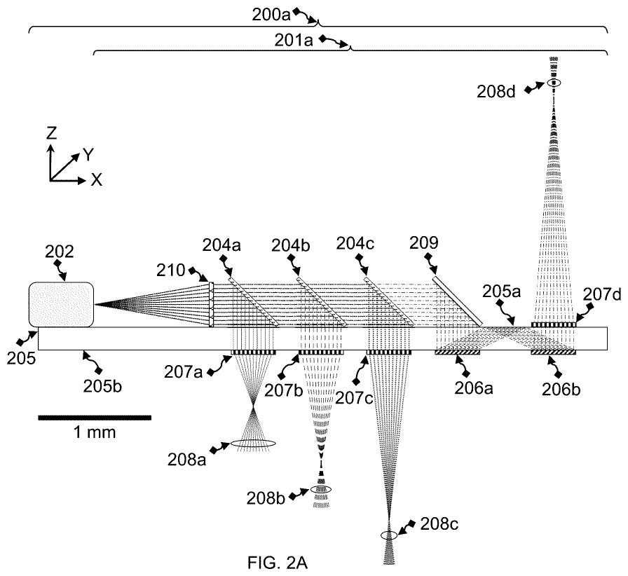

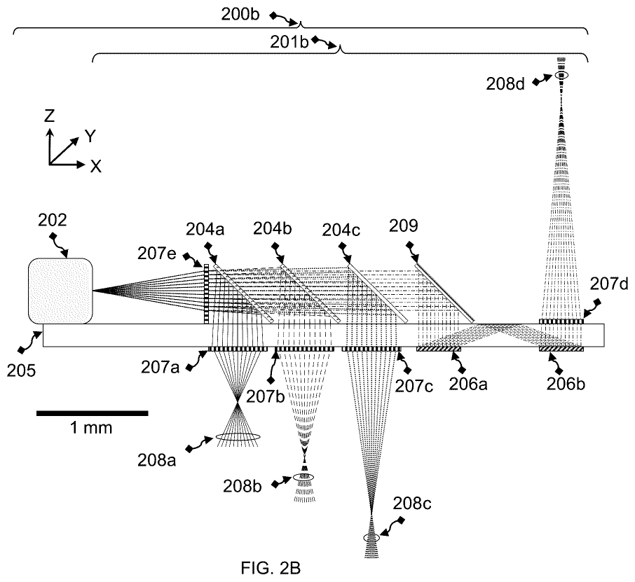

FIGS. 2A-B show multi-spectral and multi-zoom imaging embodiments using cascaded wavelength-selective reflectors.

The number of spectral channels and achievable focal lengths can be increased by stacking more WSRs and other components along the X-direction as shown in FIG. 2A-B. Notably, the internal diameter of the luminal organ (e.g., organs to be imaged or illuminated) sets a limit on the size of OIIS along the radial direction (i.e., along the Z- and Y-directions). However, this size limitation is more relaxed along the axial direction (i.e., along X-direction). Radial and axial directions are defined relative to the length of the fiber which is illustrated as a fiber 202. In reality, the fiber 202 may extend in length for centimeters or even meters along the X-direction. Fiber 202, shown in FIG. 2A, guides four spectral channels centered at, for example, first, second, third, and fourth wavelengths. In some embodiments, the first, second, third, and fourth wavelengths may be 900 nm, 1100 nm, 1300 nm, and 1500 nm, respectively. The first, second, third, and fourth spectral channels are labeled as rays 208a, 208b, 208c, and 208d, respectively. All of these rays, regardless of their wavelength, diverge after exiting the facet of fiber 202. In the ray-tracing simulations illustrated in FIGS. 2A-2B, only rays with a wavelength equal to the center wavelength of each spectral channel are shown for simplicity.

An achromatic lens 210, which may be based on metasurface design or an achromatic refractive lens, may be used to collimate rays 208a-d of all different wavelengths. After being collimated by the achromatic lens 210, the rays 208a-d encounter a series of wavelength selective reflectors that are positioned at an angle (e.g., approximately 45 degrees) relative to a first surface 205a of a substrate 205. Each of the WSRs may be configured to reflect or transmit light associated with different wavelengths. The rays 208a-d encounter a first WSR 204a after being collimated by achromatic lens 210. The WSR 204a is designed to reflect rays 208a through substrate 205 towards the first diffractive lens 207a, which may be disposed on a second surface 205b opposite a first surface 205a. The first diffractive lens 207a focuses the rays 208a at a first focal length. For example, the first focal length may be approximately 0.5 mm. The three other spectral channels (i.e., the second, third, and fourth rays 208b-d) pass through WSR 204a and continue unperturbed toward the second WSR 204b.

WSR 204b is designed to reflect rays 208b through the substrate 205 toward a second diffractive lens 207b which focuses the rays 208b at a second focal length (e.g., approximately 1 mm). WSR 204b allows rays 208c-d to pass through toward WSR 204c without interruption. WSR 204c is configured to reflect rays 208c centered at the third wavelengths toward a third diffractive lens 207c. The rays 208c are focused by the corresponding diffractive lens 207c at a third focal length (e.g., approximately 1.5 mm). WSR 204c allows rays 208d to pass through without interruption. Finally, the rays 208d corresponding with the fourth spectral channel reach mirror 209 and are re-directed toward the diffractive grating 206a. In some embodiments, a fourth WSR configured to reflect rays 208d may be used in place of a mirror.

Rays 208d are diffracted by a first diffractive grating 206a such that the diffracted rays 208a travel at angles larger than the TIR of the substrate; therefore, the rays 208d are reflected inside of the substrate when they encounter the first surface 205a of the substrate 205. The rays 208d traveling in TIR encounter a second diffractive grating 206b that again diffracts the rays 208d. Specifically, the diffractive grating 206b diffracts rays 208d toward the diffractive lens 207d to be focused at a fourth focal length (e.g., approximately 2 mm).

Utilizing gratings (e.g., diffractive gratings 206a and 206b) provides an extra degree of freedom in directing and shaping light in a small form-factor. For example, using two diffractive gratings, rays 208d may be focused from the top of the substrate (i.e., the first surface 205a), whereas other rays (i.e., rays 208a-c) are all focused below the substrate (i.e., from the second surface 205b). In the embodiment illustrated in FIG. 2A, it is demonstrated that by cascading three WSRs and one mirror, a miniaturized OIIS 201a (e.g., an OIIS having submillimeter dimensions along both Y- and Z-directions) is capable of emitting light with four different focal lengths. This OIIS 201a may be used with one spectral range at a time (e.g., by using a single spectral channel of input light) to control the focal length or may be used with a combination of the spectral channels at once (e.g., by using a multiplexed input light). Performing imaging in four spectral channels not only increases the resolution at each depth (e.g., focal length) but also enables multispectral imaging via overlapping images from each spectral channel using post-image processing techniques. When OIIS 201a is used for optical coherence tomography, depth of the image can be well beyond the focal length of each diffractive lens and each lenses' depth of focus may be designed to have overlaps across the whole spectral range to perform multispectral imaging. In addition, the number of WSRs and spectral channels may be increased to further increase the spectral range and achieve and the number of achievable focal lengths. For example, five, six, or more spectral ranges and associated WSRs and diffractive lenses may be included in an OIIS system without departing from the scope of the present disclosure.

FIG. 2B shows a similar concept as shown in FIG. 2A with the same spectral channels and focal lengths, but with a subtle difference. In OIIS 201b, the achromatic lens (210 in FIG. 2A) is replaced with a diffractive lens 207e designed at the fourth wavelength (e.g., 1500 nm wavelength). Thus, as evident from the ray-tracing simulation, only rays centered at the fourth wavelength will be perfectly collimated after passing through diffractive lens 207e. Other rays (e.g., at the first, second, and third wavelengths) are slightly diverging due to intrinsic chromatic aberration of diffractive lens 207e. For a diffractive lens, the shorter the operating wavelength, the larger difference between the operating wavelength and design wavelength (e.g., 1500 nm) and thus the larger the divergence angles. Thus, rays 208a having the smallest wavelength (e.g., a wavelength of 900 nm) will have the largest divergence angle compared to the other rays with longer wavelengths. These diverging rays may require slight changes to the design of diffractive lenses 207a, 207b, and 207c (i.e., required phase map) to make sure the diverging rays are focused down to the diffraction-limited spot. These changes can be determined by calculating a new phase map for the diffractive lenses using the ray-tracing or other available optical methods.

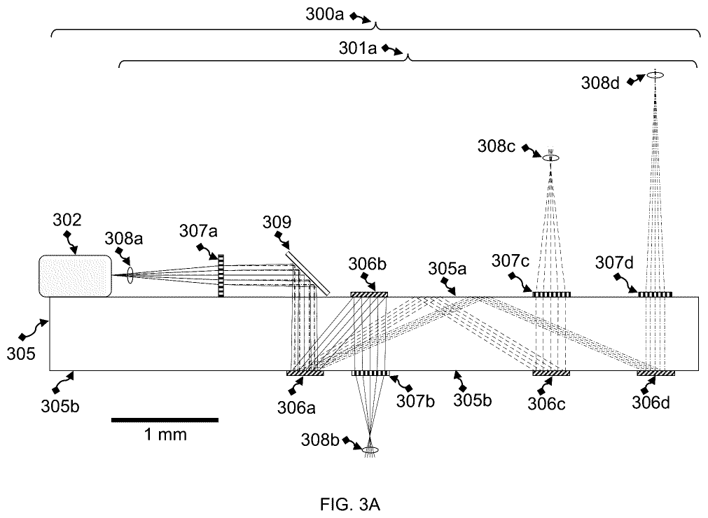

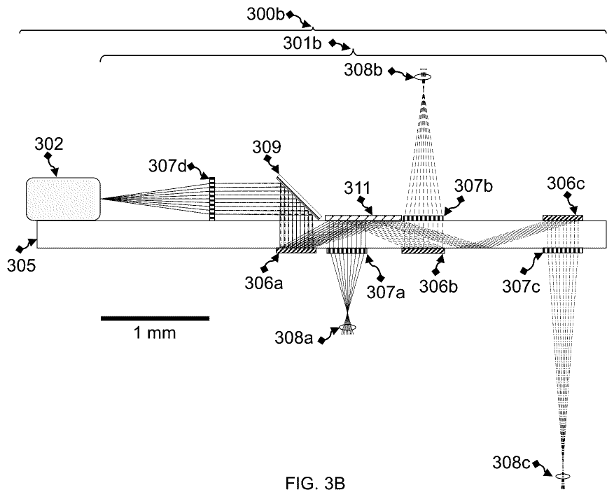

FIGS. 3A-B show multi-spectral and multi-zoom imaging and illuminating embodiments that utilize the dispersive response of diffractive gratings.

FIG. 3A shows an embodiment of endoscopic catheter 300a with multi-spectral multi-zooming OIIS 301a. In the catheter 300a, a fiber 302 delivers three spectral channels with first, second, and third center wavelengths (e.g., a first wavelength of 1000 nm, a second wavelength of 1300 nm, and a third wavelength of 1400 nm) to the OIIS 301a. Rays 308a refer to the combination of all three spectral channels. While the rays 308a begin to diverge upon leaving the fiber 302, the rays 308a are collimated by the diffractive lens 307a. Mirror 309, which may be positioned at an angle (e.g., approximately 45 degrees) relative to a top surface 305a of the substrate 305, reflects and re-directs the rays 308a toward the substrate 305. The rays 308a travel through the substrate 305 at an angle that may be substantially perpendicular to the first and second surfaces 305a, 305b of the substrate. The rays 308a encounter a first diffractive grating 306a that diffracts the rays to have angles larger than the TIR threshold of the substrate 305 so that the rays are all coupled into the substrate. It is notable that when rays' angles are larger than the TIR of the substrate, the substrate acts as a waveguide and rays can propagate inside until they are coupled out by another grating or any other appropriate components.

The diffractive grating 306a diffracts and spatially separates rays of different wavelengths as shown in FIG. 3A. As discussed above, rays 308a include rays of three different wavelengths and each wavelength is diffracted at a different angle by grating 306a. Rays 308b with the shortest wavelength (e.g., a wavelength of 1000 nm) will be diffracted into the steepest TIR angle and will be directed toward a second diffractive grating 306b. The diffractive grating 306b on the first surface of the substrate is configured to diffract rays 308b toward the diffractive lens 307b where the light is focused at a first focal length (e.g., a focal length of 0.6 mm). Two other spectral channels (i.e., rays 308c and 308d) do not impinge on the diffractive grating 306b and instead are reflected at the first surface 305a of the substrate. Rays 308c having a second wavelength larger than the first wavelength (e.g., a wavelength of 1300 nm) will be received and diffracted by a third diffractive grating 306c on the second surface 305b of the substrate. The grating 306c diffracts rays 308c toward the diffractive lens 307c where they are focused at the second focal length (e.g., a focal length of 1.2 mm) above the first surface 305a of substrate 305. Rays 308d having a third and longest wavelength (e.g., a wavelength of 1400 nm) is received and diffracted by a fourth diffractive grating 306d. The diffractive grating 306d diffracts rays 308d toward diffractive lens 307d where they are focused at a third focal length (e.g., a focal length of 2 mm).

In the OIIS 301a, the input light (e.g., ray 308a) is spatially sorted according to the different spectral channels utilizing the dispersive response of grating 306a. Other parameters, such as the thickness of the substrate 305, may also be used as a design variable in separating the different spectral channels. While diffractive lenses are described with respect to OIIS 301a, refractive lenses may be used instead of the diffractive lens without changing the functionality of the OIIS 301a.

OIIS 301a can perform imaging in the three spectral channels described, and the focal length of the system can be controlled by changing the input wavelength. For example, multiplexed input light will filter through the system as described above resulting in three light beams of different wavelengths focused at different focal lengths. Alternatively, if imaging is desired at only one of the available focal lengths, input light having a wavelength associated with that particular focal length may be provided to the OIIS 301a. While three spectral channels and associated focal lengths are described, additional channels and focal lengths may be included within the OIIS without departing from the scope of the present disclosure.

Light projected by the OIIS 301a may be reflected or otherwise scattered by a surrounding environment (e.g., an organ or tissue). At least a portion of the reflected or scattered light may be captured by the OIIS 301a via the same light paths used to deliver light to the surrounding environment but moving in the opposite direction. For example, reflected light having a first spectral channel may be captured by the diffractive lens 307b, diffracted by diffractive gratings 306b and 306a, reflected by mirror 309, shaped (e.g., converged) by diffractive lens 307a, and coupled into the fiber 302 for transmission back to an imaging system (not shown). Reflected light having second and third spectral channels may follow a similar pattern where the light moves through the light path toward the fiber for image capture. Such light-capturing capabilities are shared by all embodiments disclosed herein.

Another OIIS 301b with similar functionality is illustrated in FIG. 3B where the diffractive grating 306b (shown in FIG. 3A) is replaced with a wavelength-selective grating (“WSG”) 311. In the endoscopic catheter 300b, spatial separation between three different spectral channels (e.g., a first spectral channel centered at 1200 nm, a second spectral channel centered at 1250 nm, and a third spectral channel centered at 1300 nm) is less than that illustrated in the system 300a described above after the input light is diffracted by the diffractive grating 306a. The decreased spatial separation may be accomplished by reducing the thickness of the substrate 305, by adjusting the design of diffractive grating 306a, and/or by providing three spectral channels that are closer together in wavelength. All three spectral channels within the input light impinge on the WSG 311. The WSG 311 only diffracts the first spectral range (e.g., rays 308a centered at 1200 nm) and allows the other spectral channels to propagate undisturbed. Rays 308a impinge on diffractive lens 307a and are focused at a first focal length (e.g., a focal length of 0.6 mm). Second and third spectral channels (e.g., rays 308b and 308c, respectively) propagate through the OIIS 301b similar to the second and third spectral channels discussed with respect to OIIS 301a. Rays 308b impinge on diffractive grating 306b and are directed to diffractive lens 307b where they are focused at a second focal length (e.g., a focal length of 1.2 mm). Rays 308c impinge on diffractive grating 306c and are directed to diffractive lens 307c where they are focused at a third focal length (e.g., a focal length of 2 mm).

While specific wavelengths and focal lengths are provided as examples for purpose of description, one of skill in the art will appreciate that other wavelengths and/or focal lengths may be selected without departing from the scope of the present disclosure.

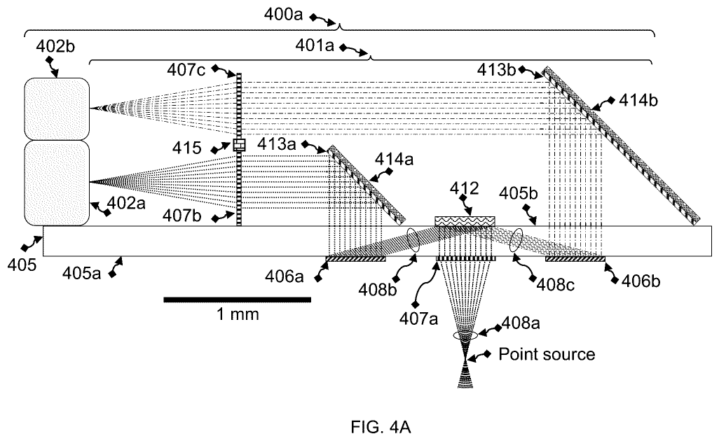

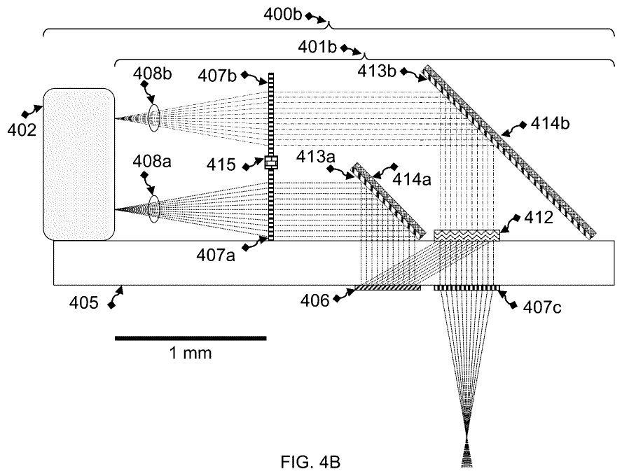

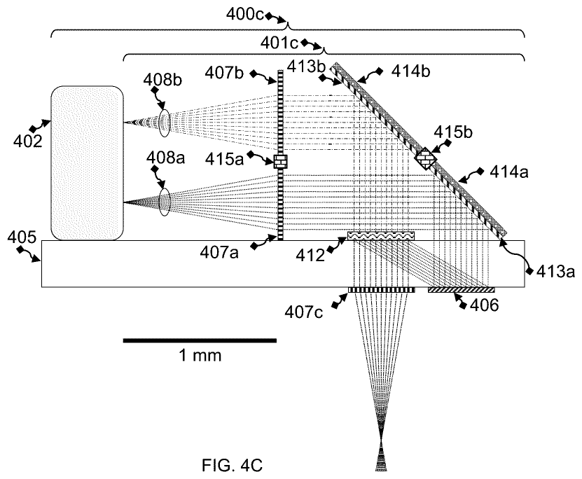

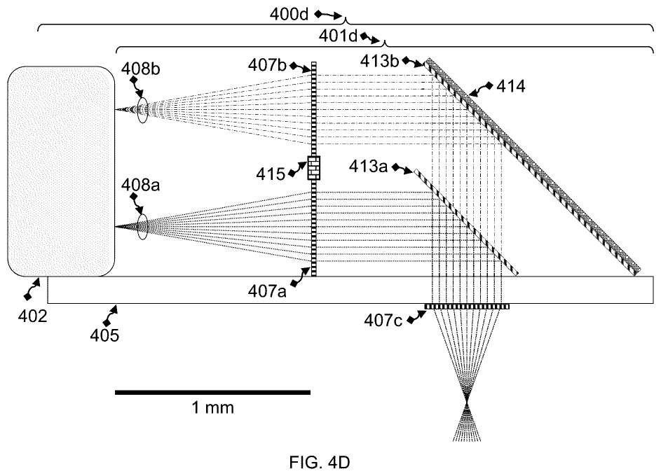

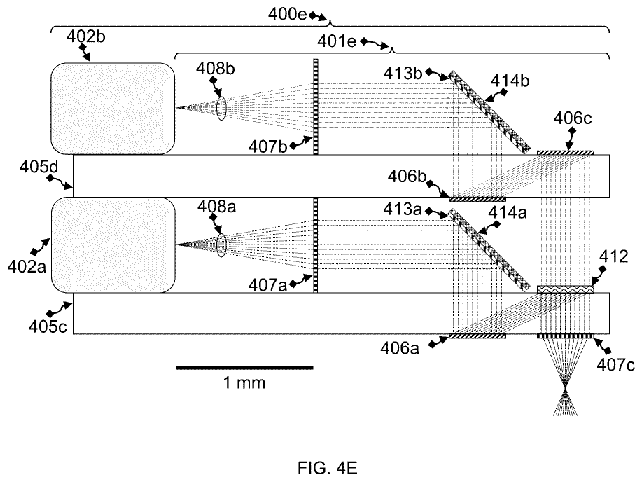

FIGS. 4A-E show five embodiments of miniaturized polarization-resolved imaging and illuminating systems.

Embodiments illustrated in FIGS. 1-3 have been described with respect to their imaging capabilities. That is, each OIIS is described by tracing light from the facet of fiber, through several components, and eventually to the focal spot. Each OIIS is a reciprocal system meaning that the same system will collect the light from the scene (e.g., an object which is being imaged, such as a tissue, in the case of medical imaging) and send it back to the fiber to be delivered to the image processing module (not shown) to form images.

In the ray-tracing simulation shown in FIG. 4A, endoscopic catheter 400a having OIIS 401a will be described starting from a point source located at the focal spot of diffractive lens 407a. One can see this point source as an infinitesimal part of a tissue that has already been illuminated by the same OIIS 401a and now scatters light upward (i.e., toward the 407a) and downward. The upwardly scattered rays, labeled rays 408a, are collected by diffractive lens 407a and then are collimated toward Polarization-Selective Grating (“PSG”) 412. The polarization states of rays 408a can be decomposed into two orthogonal components: Polarization #1 (P₁) depicted by rays 408b and Polarization #2 (P₂) depicted by rays 408c. The PSG 412 diffracts light into different directions (e.g., spatially separates) based on polarization. For example, PSG 412 diffracts rays 408b with polarization P₁ toward the diffractive grating 406a and rays 408c having polarization P₂ toward the diffractive grating 406b. Diffractive grating 406a diffracts rays 408b and rays 408b outcouple from the substrate 405 at an angle substantially perpendicular to first and second surfaces 405a, 405b of the substrate. Rays 408b travel toward the Polarization-Selective Reflector (“PSR”) 413a, which is oriented in such a way (e.g., at approximately 45 degrees relative to the first surface) that it reflects the rays 408b at an angle (e.g., to a direction that is approximately 0 degrees relative to the second surface 405b) toward a fiber. Finally, 408b rays are coupled into a first fiber 402a via a diffractive lens 407b that converges the rays 408b to a focal spot on a facet of a fiber core within first fiber 402a.

Rays 408c take a different path toward a second fiber 402b via diffractive grating 406b, a second PSR 413b, and diffractive lens 407c. The PSR 413b is oriented in such a way (e.g., at approximately 45 degrees relative to the second surface 405b) that it reflects light rays 408c toward a fiber at an angle (e.g., to a direction approximately 0 degrees relative to the second surface 405b). In some embodiments, the PSR 413b only reflects light having a polarization P₂ in order to prevent any stray P₁ light from entering the P₂ light pathway at the fiber. To further reduce the likelihood of any P₁ polarization light incoupling to the P₂ pathway, an absorptive polarizer 414b may be applied to a back surface of PSR 413b. The absorptive polarizer ensures that if there is any other polarization component than intended polarization, it will be absorbed to prevent it from continuing to propagate through the system along the incorrect pathway. A similar absorptive polarizer component 414a may be used on the PSR 413a to absorb stray P₂ polarization light within the P₁ polarization light pathway. The polarization direction of the absorptive polarizers is orthogonal to the corresponding PSRs that they are stacked against. In some embodiments, PSR 413b and absorptive polarizer 414b may be replaced by a single metallic or dielectric mirror without departing from the scope of the present disclosure. In some embodiments, the PSR 413a and absorptive polarizer 414a may also be replaced with a single metallic or dielectric mirror.

The OIIS 401a is capable of performing polarization-resolved imaging since OIIS 401a spatially separates two orthogonal polarizations of light coming from the imaged object and sends them to two fibers which will be eventually received by a processing module that may include a camera or optical sensors (not shown here) to form images. In some embodiments, the P₁ and P₂ polarizations of light may be coupled into two different fiber cores within a single fiber.

Another embodiment capable of polarization-resolved imaging is shown in FIG. 4B. Endoscopic catheter 400b having OIIS 401b is a modified version of the embodiment shown in FIG. 4A. The OIIS 401b includes one less component (i.e., one less diffractive grating, such as diffractive grating 406b from FIG. 4A). The diffractive lens 407c of OIIS 401b receives scattered or reflected light from the imaged object; the received light includes both P₁ and P₂ polarization components. The light impinges on PSG 412 where a first polarization (e.g., P₁ polarization) light is diffracted toward diffractive grating 406 and follows a path as discussed with respect to FIG. 4A. PSG 412 is configured such that light having a second polarization (e.g., P₂ polarization) is not diffracted, but rather, passes through the PSG 412 without interruption. The P₂ light then encounters PSR 413b and travels through a pathway similar to that described with respect to FIG. 4A.

In addition to reducing the number of components on the OIIS 401b, the number of components within the endoscopic catheter 400b may be reduced by replacing the two fiber configurations used in 400a with one fiber having two cores in 400b. The system can be further simplified by grouping PSRs 413a, 413b and absorptive polarizers 414a, 414b. This embodiment is shown in FIG. 4C.

Another alternative embodiment for polarization-resolved imaging is shown FIG. 4D. In the endoscopic catheter 400d having OIIS 401d, rays collected by diffractive lens 407c are sorted by the PSR 413a which reflects P₁ polarization (i.e., rays 408a) and lets the rays with orthogonal polarization (i.e., P₂, shown by rays 408b) pass through. The reflected rays 408a are focused by a first diffractive lens 407a and are coupled into a first core of the fiber 402. PSR 413b receives and is configured to reflect P₂ polarization light. Thus, the rays 408b are reflected by second PSR 413b toward the diffractive lens 407b and are coupled into a second fiber core. An absorptive polarizer 414 may be included on the PSR 413b to absorb any stray P₁ polarization light. Rays 408a and 408b travel along with their respective fiber cores to the processing module for image processing.

Another embodiment for polarization-resolved imaging is shown in FIG. 4E. The system 400e having OIIS 401e includes a first and second fiber 402a and 402b located on a first and second substrate 405c and 405d, respectively. In this embodiment, light coupled out of fibers 402a, 402b is already polarized; for example, rays 408a may have P₁ polarization, and rays 408b may have P₂ polarization. In alternative embodiments, if the light exiting fibers 402a, 402b is unpolarized, one or more polarizer components (not shown) may be placed between the end of each fiber 402a, 402b, and diffractive lenses 407a and 407b and/or may be placed between the diffractive lenses 407a, 407b, and PSRs 413a, 413b, respectively. Light received by the first PSR 413a may be a first polarization (e.g., P₁ polarization) while light received by the second PSR 413b may be a second polarization (e.g., P₂ polarization).

Rays 408b coupled out of the second fiber 402b are collimated by the diffractive lens 407b and are reflected by PSR 413b toward the diffractive grating 406b. An absorptive polarizer 414b may be included on the PSR 413b as shown. The rays 408b are diffracted by diffractive gratings 406b and 406c toward PSG 412. The PSG 412 allows rays 408b (i.e., rays having P₂ polarization) to pass through undisturbed. The rays 408b combine with rays 408a and are focused by diffractive lens 407c at a focal length (e.g., a focal length of 0.4 mm). Rays 408a with orthogonal polarization (e.g., P₁ polarization), relative to 408b, couple out of bottom fiber 402a and are collimated by diffractive lens 407a. The rays 408a are re-directed toward diffractive lens 407c via PSR 413a, diffractive grating 406a, and PSG 412. PSR 413a may include an absorptive polarizer 414a disposed thereon. PSG 412 is configured to diffract light having polarization P₁; thus, rays 408a are diffracted by PSG 412 toward the diffractive lens 407c where they are focused at the focal length along with rays 408b. Due to the reciprocity of the system 400e when collecting light for imaging, each ray scattered by the imaged object is sorted based on polarization and is coupled to a corresponding fiber core. The light collected in both fiber cores is sent to the image processing module (not shown) to perform polarization-resolved imaging.

FIGS. 4A-4E discussed above illustrate example embodiments of endoscopic catheters capable of performing polarization-resolved imaging with small form-factor OIIS that utilize flat components. One of skill in the art will appreciate that some of the components may be replaced by refractive or metasurface counterparts without departing from the scope of the present disclosure. For example, one or more diffractive lenses may be replaced with refractive lenses. Also, several other embodiments that are not described in detail herein may be designed by combining or altering various features discussed with respect to FIGS. 4A-4E. Notably, each of the described embodiments has advantages that may be particularly well-suited to an application depending on imaging or illuminating requirements. For example, in the embodiment shown in FIG. 4A rays will interact with three polarization components (PSG, PSR, and absorptive polarizer). The first PSG 412 sorts the rays based on their polarization and later each of these polarized rays will interact with a PSR that only reflects a specific polarization. If there is any residual unwanted polarization in each optical path, it will be absorbed by the absorptive polarizers which increases the signal-to-noise ratio of the imaging system. Other embodiments, such as system 400d shown in FIG. 4D may benefit from increased efficiency. In particular, the embodiment illustrated in FIG. 4D requires fewer components and may result in less optical loss of light due to absorption or other imperfection of each components.

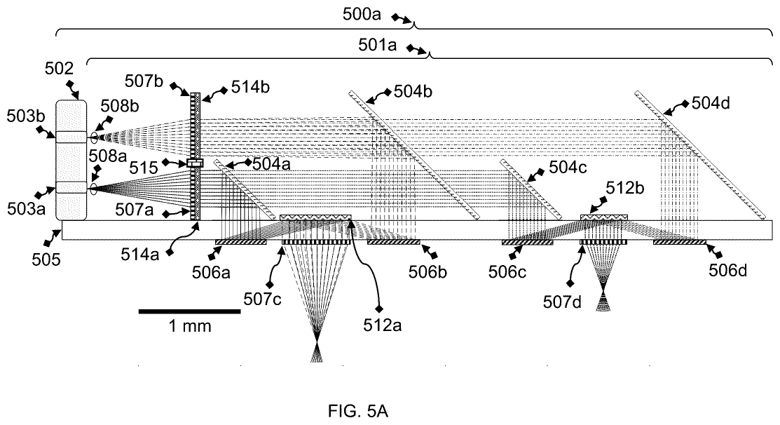

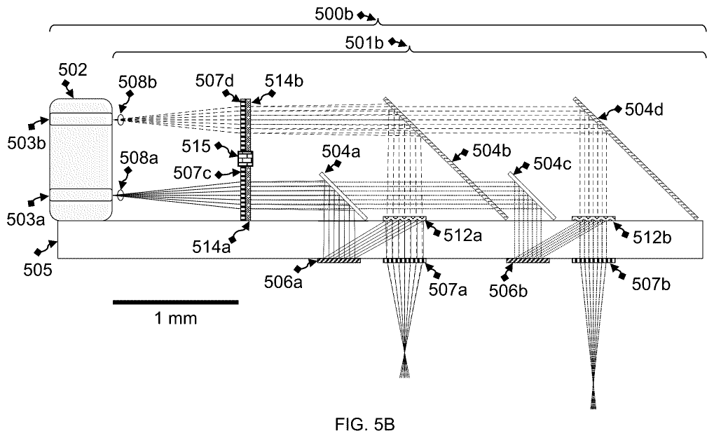

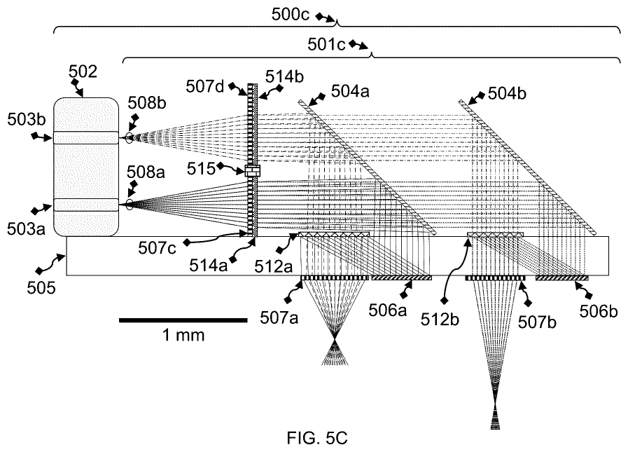

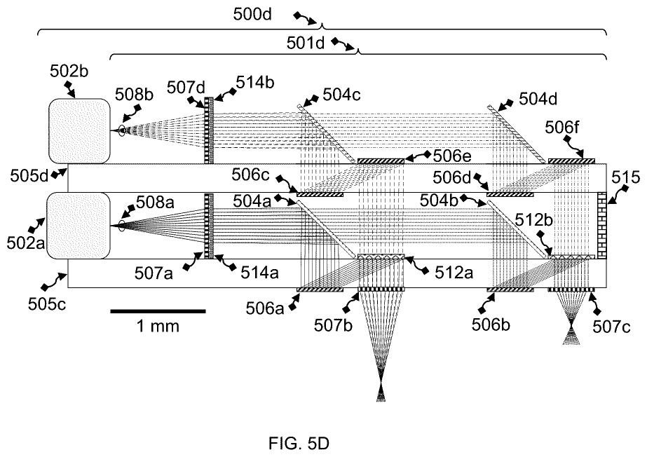

Turning to FIGS. 5A-D, multifunctional optical imaging, and illuminating embodiments are illustrated. In particular, four embodiments of multifunctional OIIS are shown wherein the concepts of multispectral, multi-zoom, and polarization-resolved imaging are combined in a single system. Referring initially to FIG. 5A, an endoscopic catheter 500a is shown having an OIIS 501a configured to perform multi-spectral, multi-zoom, and polarization-resolved imaging simultaneously. This embodiment can be seen as a fusion of embodiments shown in FIG. 2B and FIG. 4A. In this endoscopic catheter 500a, fiber 502 has two cores 503a, 503b and both of the cores carry two spectral channels centered at a first wavelength and a second wavelength (e.g., 1200 nm and 1300 nm, respectively). Rays 508a coupled out of the bottom core 503a are collimated by diffractive lens 507a and become linearly polarized (e.g., are polarized with P₁ polarization) by passing through the absorptive polarizer 514a adjacent to the diffractive lens 507a. While the absorptive polarizer is shown stacked against the diffractive lens 507a on a side opposite the fiber 502, the absorptive polarizer may be spaced apart from the diffractive lens 507a and/or may be placed before or after the diffractive lens 507a along the light path. The rays 508a impinge on a WSR 504a where a first portion (e.g., the first spectral channel centered at, for example, 1200 nm) of the rays 508a are reflected toward the substrate 505. The first portion of rays 508a are diffracted by diffractive grating 506a and then are diffracted by PSG 512a toward the diffractive lens 507c where they are focused at a first focal length (e.g., at a focal length of 1 mm). Similarly, a first portion (e.g., a first spectral channel centered at, for example, 1200 nm) of the rays 508b from the top core are focused at the same first focal spot after interacting with diffractive lens 507b, absorptive polarizer 514b, WSR 504b, diffractive grating 506b, PSG 512a, and diffractive lens 507c. This optical path shows how the light will be focused on the object; rays scattered by the object will also take the same path in reverse through the OIIS 501a to be coupled-in to the fiber for image processing.

A second portion of the rays 508a (e.g., a second spectral channel with wavelength centered at, for example, 1300 nm) from the bottom core 503a passes through WSR 504a and 504b undisturbed. This second portion of the rays 508a are reflected by WSR 504c toward diffractive grating 506c which in turn diffracts the rays toward PSG 512b. PSG 512b diffracts the second portion of rays 508a toward a diffractive lens 507d which focuses the light at a second focal length (e.g., a focal length of 0.5 mm). Similarly, a second portion (e.g., a second spectral channel with wavelength centered at, for example, 1300 nm) of the rays 508b from the top core 503b passes through WSR 504b undisturbed. The second portion of rays 508b impinge on WSR 504d where they are reflected toward the substrate 505 and a diffractive grating 506d disposed thereon. The diffractive grating 506d diffracts the second portion of rays 508b toward PSG 512b which in turn diffracts the light toward diffractive lens 507d. The diffractive lens 507d outcouples and focuses the light at the second focal length.

Thus, the OIIS 501a is capable of performing polarization-resolved imaging at two different focal lengths, where the focal length of the imaging is controlled by the center wavelength of the spectral channel of the input light. Additional fiber cores, diffractive lenses, WSRs, PSGs, and diffractive gratings may be added in sequence to the system to increase the number of spectral channels and associated focal lengths.

FIG. 5B shows a system 500b having an OIIS 501b which can perform multi-spectral, multi-zoom, and polarization-resolved imaging in a single embodiment. This embodiment combines concepts described above with respect to FIG. 2B and FIG. 4B embodiments. OIIS 501b has two fewer components (i.e., diffractive gratings) compared to OIIS 501a which may result in reduced system complexity and reduced cost associated with fabrication and assembly. System 500b includes a fiber 502 having a first core 503a and a second core 503b. Cores 503a and 503b may each carry light having two spectral channels (e.g., centered at 1100 nm and 1300 nm). Light from first core 503a travels through and is collimated by diffractive lens 507c. An absorptive polarizer 514a ensures that light passing through has only a single polarization (e.g., P₁ polarization). The light encounters WSR 504a where a first portion (e.g., P₁ polarization, spectral channel centered at 1100 nm) is reflected toward the substrate 505 and a diffractive grating 506a disposed thereon. Diffractive grating 506a directs the first portion of rays 508a to the PSG 512a where it is again diffracted toward diffractive lens 507a. The lens 507a focuses the light at a first focal length (e.g., a focal length of 0.75 mm). A second portion of rays 508a (e.g., P₁ polarization, spectral channel centered at 1300 nm) pass through the WSRs 504a, 504b undisturbed and are reflected by WSR 504c toward diffractive grating 506b. Diffractive grating 506b directs the second portion of rays 508a toward PSG 512b where it is diffracted to diffractive lens 507b. Lens 507b focuses the light at a second focal length (e.g., a focal length of 1 mm).

Rays 508b from the second core 503b pass through a diffractive lens 507d and a polarizer 514b. The polarizer 514b causes the rays 508b to have a second polarization (e.g., P₂ polarization). The diffractive lens 507c and polarizer 514a are separated from the diffractive lens 507d and polarizer 514b by a spacer 515. A first portion of the rays 508b (e.g., P₂ polarization, spectral channel centered at 1100 nm) are reflected by WSR 504b toward the PSG 512a. PSG 512a permits the P₂ polarized first portion of rays 508b to pass therethrough where it impinges on diffractive lens 507a. The light is focused at the first focal length. A second portion of rays 508b (e.g., P₂ polarization, spectral channel centered at 1300 nm) passes through the WSR 504b and are reflected by WSR 504d toward second PSG 512b. The PSG 512b permits the P₂ polarized second portion of rays 508b to pass therethrough toward the diffractive lens 507b. Diffractive lens 507b focuses the second portion of rays 508b at the second focal length.

The number of components in the endoscopic catheter can be further reduced using the embodiment shown in FIG. 5C where the OIIS 501c includes two fewer WSRs compared to the OIIS 501b. This embodiment can be seen as a fusion of the OIISs shown in FIG. 2B and FIG. 4C. Diffractive lenses 507a and 507b (FIG. 5C) work at spectral channels centered at a first wavelength (e.g., 1100 nm) and a second wavelength (e.g., 1300 nm) resulting in a light focused at a first focal length (e.g., a focal length of 0.5 mm) and a second focal length (e.g., 1 mm), respectively. Rays 508a from a first fiber core 503a travel through a diffractive lens 507c and polarizer 514a where it is polarized with a first polarization (e.g., P₁ polarization). The light impinges on a first WSR 504a where a first portion (e.g., spectral channel centered at 1100 nm) is reflected toward a diffractive grating 506a on the substrate 505. Diffractive grating 506a diffracts light toward a first PSG 512a which is configured to diffract P₁ polarized light toward a first diffractive lens 507a. The first portion of rays 508a is focused at the first focal length. A second portion of the rays 508a (e.g., spectral channel centered at 1300 nm) passes through the first WSR 504a and is reflected by the second WSR 504b toward a second diffractive grating 506b on the substrate 505. The diffractive grating 506b diffracts the light toward a second PSG 512b which is configured to diffract P₁ polarized light toward a second diffractive lens 507b. The diffractive lens 507b focuses the second portion of rays 508a at the second focal length.

Rays 508b from the second fiber core 503b travel through a diffractive lens 507d and polarizer 514b where they are polarized with a second polarization (e.g., P₂ polarization) opposite of the first polarization. The light impinges on the first WSR 504a where a first portion (e.g., spectral channel centered at 1100 nm) is reflected toward the PSG 512a which is configured to transmit light with polarization P₂. Thus, the first portion of rays 508b passes through PSG 512a undisturbed toward diffractive lens 507a where it is focused at the first focal length. A second portion of rays 508b (e.g., spectral channel centered at 1300 nm) passes through the WSR 504a and is reflected by the second WSG 504b toward the second PSG 512b. PSG 512b is configured to transmit light with polarization P₂, thus, the second portion of rays 508b passes through the PSG 512b undisturbed toward diffractive lens 507b. The lens 507b focuses the second portion of rays 508b at the second focal point. Light reflected from the environment (e.g., a surrounding tissue) enters the OIIS 501c via diffractive lenses 507a, 507b and travels in reverse through the light pathways described above for imaging purposes.

Referring now to FIG. 5D, a catheter system 500d is shown having an OIIS 501d. The OIIS 501d combines concepts described above with respect to FIG. 2B and FIG. 4E. The OIIS 501d (shown in FIG. 5D) is configured to provide multispectral, multi-zoom, and polarization-resolved imaging at two spectral channels (i.e., centered 1200 nm and 1300 nm) with two different focal lengths (i.e., focal lengths of 1 mm and 0.4 mm). Light rays 508a exiting a first fiber 502a may include a first and a second spectral channel. Rays 508a are collimated and polarized by diffractive lens 507a and polarizer 514a, respectively. The rays 508a may all have a first polarization (e.g., P₁ polarization). Rays 508a encounter WSR 504a configured to reflect a first spectral channel and transmit the second spectral channel. Thus, the first spectral channel is reflected toward a first diffractive grating 506a on a first substrate 505c where it is diffracted within the substrate toward a PSG 512a configured to diffract light having the first polarization (e.g., P₁ polarization). Rays 508a having the first spectral channel are focused by diffractive lens 507b at a first focal point (e.g., 1 mm) as they exit the first substrate toward an environment. The second spectral channel continues through WSR 504a toward a second WSR 504b configured to reflect light at the second spectral channel. Thus, light rays at the second spectral channel are reflected toward a second diffractive grating 506b on the first substrate which diffracts the light toward a second PSG 512b configured to diffract light having the first polarization (e.g., P₁ polarization). Light is diffracted toward diffractive lens 507c where it is focused at a second focal point (e.g., 0.4 mm) after exiting the first substrate.

Rays exiting the second fiber 502b follows a separate but similar path. Rays 508b, including light centered at the first and second spectral channels, pass through diffractive lens 507d and polarizer 514b where they are approximately collimated and polarized with a second polarization (e.g., P₂ polarization), respectively. Light at the first spectral channel is reflected by WSR 504c where it is diffracted by diffractive grating 506c on the second substrate 505d. Diffractive grating 506c diffracts the light toward diffractive grating 506e which diffracts the light out of the second substrate 505d toward PSG 512a. The first spectral channel of light rays 508b may exit the second substrate 505d traveling substantially perpendicular to and aligned with PSG 512a. Because this light is P₂ polarized, it transmits through PSG 512a toward diffractive lens 507b where it is focused, along with the first spectral channel of rays 508a, at the first focal point. The second spectral channel of rays 508b passes through WSR 504c where they are reflected by WSR 504d configured to reflect light at the second spectral channel. The light is diffracted by diffractive grating 506d on the second substrate 505d toward diffractive grating 506f which diffracts light having the second polarization P₂. The second spectral channel of light then exits the second substrate approximately perpendicular to and aligned with PSG 512b, which is configured to transmit light having second polarization P₂ toward the diffractive lens 507c where it is focused, along with the second spectral channel of rays 508a, at the second focal length. Spacer 515 is placed between first substrate 505c and second substrate 505d to facilitate their assembly and angular alignment. In the system 500d, first substrate 505c and second substrate 505d are substantially parallel.

As discussed with prior embodiments, the system 500d is a reciprocal system and is configured to capture light scattered or reflected by the surrounding environment (e.g., tissues and organs). Reflected or scattered light enters the system OIIS 501d through diffractive lenses 507b, 507c and travels in reverse through the light paths described above. Thus, light having a first polarization P₁ at the first and second spectral lengths is captured by first fiber 502a, and light having a second polarization P₂ at the first and second spectral lengths is captured by second fiber 502b. In the first fiber 502a, light at the first spectral channel is focused at the first focal length and light at the second spectral channel is focused at the second focal length. Similarly, in the second fiber 502b, light at the first spectral channel is focused at the first focal length and light at the second spectral channel is focused at the second focal length.

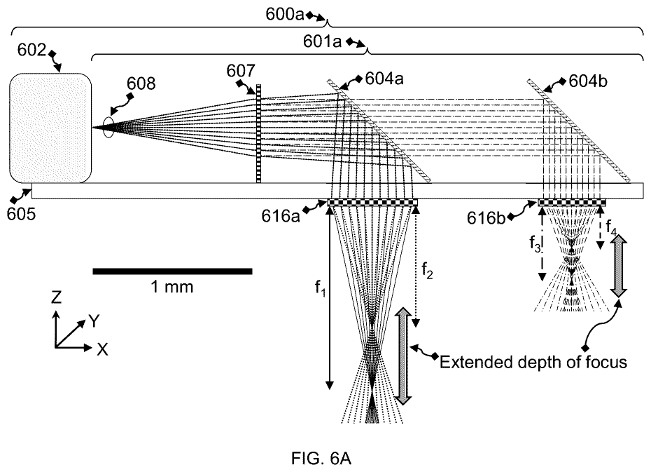

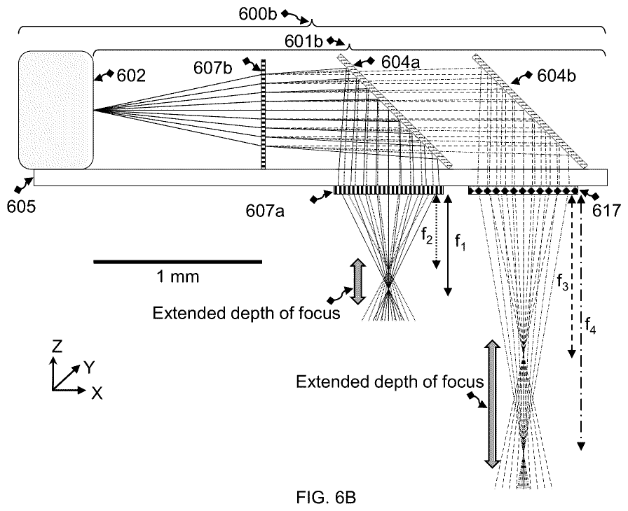

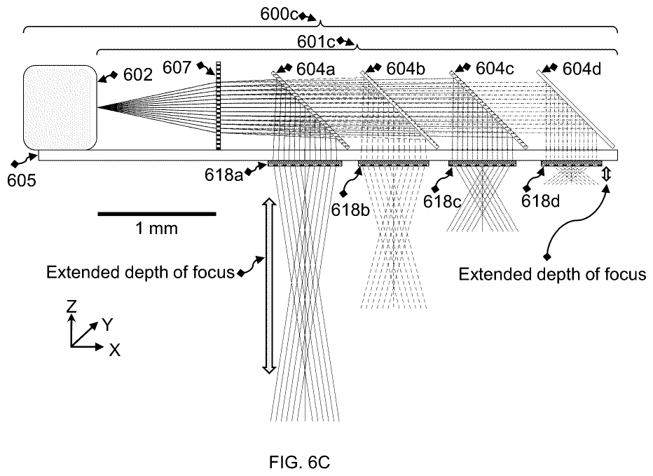

FIG. 6A-C show three embodiments configured to extend the depth of focus of Optical Imaging and Illuminating Systems (“OIIS”).

In 3-dimensional medical imaging, depth information is very important for diagnosis and/or treatment. Generally, in an OCT system, resolution in the radial direction (e.g., depth into tissues of a luminal organ along the optical axis of the OIIS) is determined by the interferometry process; however, collection efficiency of OIIS depends at least in part on the depth of focus of the OIIS. Collection efficiency is defined as how much of the signal (i.e., light scattered by the tissue) at different depths can be collected by OIIS and sent to the image processing module to form images and perform analysis. However, there is a trade-off between lateral resolution (e.g., imaging resolution in the plane perpendicular to the optical axis) and its depth of focus. For example, if the NA of the OIIS increases, it focuses light to a smaller spot that may result in higher lateral resolution. However, increasing the NA also generally results in the reduction of depth of focus. Three embodiments configured to extend the depth of focus while maintaining high lateral resolution are described.

A catheter system 600a is shown in FIG. 6A. The system 600a includes an OIIS 601a that makes use of Polarization Selective Diffractive Lenses (“PSDL”) to extend the depth of focus of OIIS. PSDLs diffract light differently depending on polarization; for example, light having a first polarization may be diffracted toward a first focal spot while light having a second polarization may be diffracted toward a second focal spot different from the first focal spot. Thus, a beam made up of light having two different polarizations results in portions of the beam being focused at two different focal lengths.

A fiber 602 (for simplicity the ferrule which holds fiber 602 is not shown here) receives two spectral channels from the source (not shown) wherein the first spectral channel is centered at a first wavelength (e.g., 800 nm) and a second spectral channel is centered at a second wavelength (e.g., 1300 nm). The first and second spectral channels are included in rays 608 illustrated exiting the fiber 602 toward a diffractive lens 607. In some embodiments, one of the spectral channels (e.g., the second spectral channel) is roughly collimated by diffractive lens 607 while the other spectral channel (e.g., the first spectral channel) is shaped toward a more collimated beam but is not collimated. The difference in light shaping between the two spectral channels may occur because diffractive lens 607 is designed to collimate one spectral channel, while rays at the other spectral channel will not be perfectly collimated by the diffractive lens due to chromatic dispersion. Both spectral channels contain two orthogonal polarizations (e.g., P₁ and P₂ polarization). The rays 608 having the first spectral channel are reflected by WSR 604a designed to reflect the first spectral channel and transmit the second spectral channel. The reflected rays travel toward a first PSDL 616a on the substrate 605. The first PSDL 616a is configured to focus a portion of the first spectral channel having polarization P₁ (as illustrated by solid lines) at first focal length (e.g., f₁=1.2 mm) and is configured to focus a portion of the first spectral channel having polarization P₂ (as illustrated by dotted lines) at a second focal length (e.g., f₂=0.8 mm). Focusing light centered at the same spectral channel at both first and second focal lengths extends the depth of focus of OIIS 601a at the spectral channel. Light that is scattered by the imaged object may be captured more efficiently by the OIIS 601a if it is within a certain distance (depth of focus of each focal point) of either the first or second focal length. Depth of focus (DOF) can be defined as follows:

$$DOF = ∓ \frac{λ \sqrt{{n}^{2} - {NA}^{2}}}{2 {NA}^{2}} \qquad ( 1 )$$

where n is the refractive index of the medium, λ is the wavelength of light, and NA is the numerical aperture. This DOF value determines the distance in the vicinity of the focal spot along the optical axis where the image stays focus. In an OIIS with two focal lengths, by appropriate design of parameters (e.g., wavelength, numerical aperture), it may be beneficial to have a DOF of each focal spot overlap such that the OIIS has an extended depth of focus which goes beyond what is conventionally possible.

The second spectral channel passes through WSR 604a undisturbed and is reflected toward a second PSDL 616b by a second WSR 604b. Rays with P₁ polarization (as illustrated by dashed lines) are focused at a third focal length (e.g., f₃=0.5 mm) and rays having P₂ polarization (as illustrated by dash-dotted lines) are focused at a fourth focal length (e.g., f₄=0.3 mm). As discussed above with respect to the first and second focal lengths, being able to use collected light within a range of the third and fourth focal lengths extends the depth of focus of OIIS 601a at the second spectral channel.

In an OCT imaging system or any other type of imaging system, excitation light (e.g., light delivered to a surrounding environment such as tissue) may have substantial bandwidth meaning that it is not a single wavelength with very narrow bandwidth. The excitation light may come from LEDs, swept-source laser, VCSELs, supercontinuum sources, superluminescent diodes, any other type of light source with adjustable center wavelength and/or tunable bandwidth. By designing diffractive lenses with tailored chromatic dispersion, the broad bandwidth of input light may be used to extend the depth of focus of an OIIS system. The focal length of each diffractive lens is assumed to be a function of wavelength as related in Equation 2:

$$f = {C ( \frac{1}{λ} )}^{m} \qquad ( 2 )$$

In Equation 2, f is the focal length, C is a constant, λ is the wavelength, and m is an integer value. Referring now to FIG. 6B, catheter system 600b includes an OIIS 601b that illustrates two examples of diffractive lenses that can be described by Equation 2 above. A first diffractive lens 607a is shown where m=1 (e.g., a normal diffractive lens) and a second diffractive lens 617 is shown with m=3 (e.g., a super-dispersive diffractive lens). In this embodiment, fiber 602 outcouples a first spectral channels (e.g., centered at 1000 nm) and a second spectral channel (e.g., centered at 1300 nm). Each of the channels has a spectral bandwidth with a Full Wave Half Maximum (“FWHM”) (e.g., each spectral channel may have a FWHM of 200 nm). In ray-tracing simulations, like that shown in FIG. 6B, each ray may be modeled having a single wavelength for simplicity. Four different rays with first, second, third, and fourth wavelengths are shown. In some embodiments, the first, second, third, and fourth wavelengths may be approximately 900 nm (solid lines), 1100 nm (dotted lines), 1200 (dash-dotted lines), and 1400 nm (dashed lines), respectively. All rays exit fiber 602 and pass through diffractive lens 607b where they are approximately collimated. The lens 607b may be designed to perfectly collimate light having a wavelength within the range of wavelengths covered by first through fourth rays. For example, lens 607b may be designed to perfectly collimate light having 1300 nm wavelength. Rays having wavelengths different from the designed wavelength are not perfectly collimated: they may either slightly diverge or converge after the lens 607b.

In this example, first and second rays with wavelength 900 nm and 1100 nm are reflected toward the diffractive lens 607a by the WSR 604a. The WSR 604a may be positioned at an angle (e.g., approximately 45 degrees) with respect to a top surface of the substrate 605 such that reflected rays enter the substrate approximately perpendicular to the top surface. The diffractive lens 607a is a normal diffractive lens where m=1 in Equation 2. Therefore, lens 607a focuses the first and second rays at first and second focal lengths (e.g., f₁=0.611 mm and f₂=0.5 mm), respectively, thereby expanding the depth of focus of OIIS 601b at the first and second spectral channels. Third and fourth rays (e.g., rays with wavelengths of 1200 nm and 1400 nm, respectively) pass through WSR 604a and are reflected by a second WSR 604b toward super-dispersive diffractive lens 617. The focal lengths of this super-dispersive diffractive lens 617 follow Equation 2 with m=3. With a super-dispersive diffractive lens, a larger focal length shift is achieved by changing the wavelength. This effect is illustrated in the ray-tracing simulation where the third ray is focused at a third focal length (e.g., 1.588 mm at wavelength 1200 nm) and the fourth ray is focused at a fourth focal length (e.g., 1 mm at wavelength 1400 nm). Thus, the super-dispersive diffractive lens may be used to further expand the depth of focus of OIIS 601b at the third and fourth spectral channels.

Referring now to FIG. 6C, catheter system 600c having OIIS 601c illustrates an embodiment wherein the depth of focus is extended by utilizing an axicon to focus the light. In OIIS 601c, four axicons 618a-618d are assumed to generate Jo Bessel beams but have different numerical apertures (“NA”). In the system 600c, fiber 602 carries four spectral channels centered at first, second, third, and fourth wavelengths (e.g., 1000 nm, 1100 nm, 1200 nm, and 1300 nm, respectively). These spectral channels are collimated, or are approximately collimated, by the diffractive lens 607. The first rays having the first wavelength are reflected by WSR 604a toward the substrate 605 and are focused by a first axicon 618a. This axicon is designed at the first wavelength (e.g., a wavelength of 1000 nm) and has first numerical aperture (e.g., NA₁=0.15). This relatively small NA results in relatively large (e.g., millimeter scale) depth of focus as shown in FIG. 6C. The second through fourth rays pass through the WSR 604a. Second rays are reflected by second WSR 604b and are focused by the second axicon 618b. In some embodiments, the second axicon 618b has a second NA larger than the first NA (e.g., NA₂=0.25). By increasing the NA, a smaller focal spot is achieved. The smaller focal spot provides better resolution for imaging at the expense of reducing the depth of focus.

Third and fourth rays pass through second WSR 604a undisturbed. Third rays are reflected by third WSR 604c and while fourth rays pass through third WSR 604c and are reflected by fourth WSR 604d. Third rays are focused by third axicon 618c having a third NA (e.g., NA₃=0.5) and a fourth axicon 618d having a fourth NA (e.g., NA₄=0.8), respectively. With increasing NA, the depth of the focal spot is reduced, and resolution is increased. Overall, the depth of focus of OIIS 601c is increased by utilizing one or more axicons for focusing. One or more depths of focus may be selected for imaging by changing the spectral channel of the input signal; thus, the OIIS 601c provides adjustable depth of focus and NA.

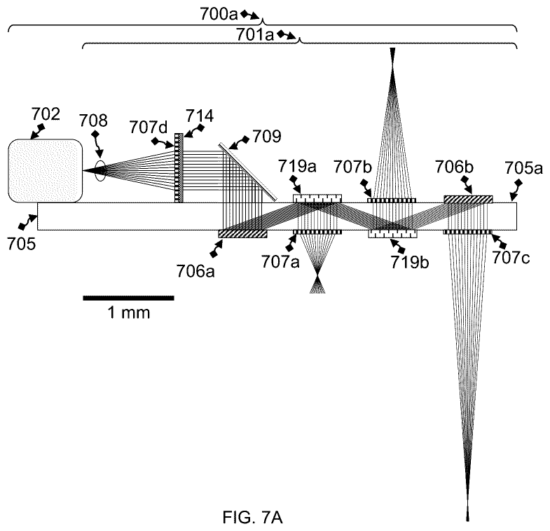

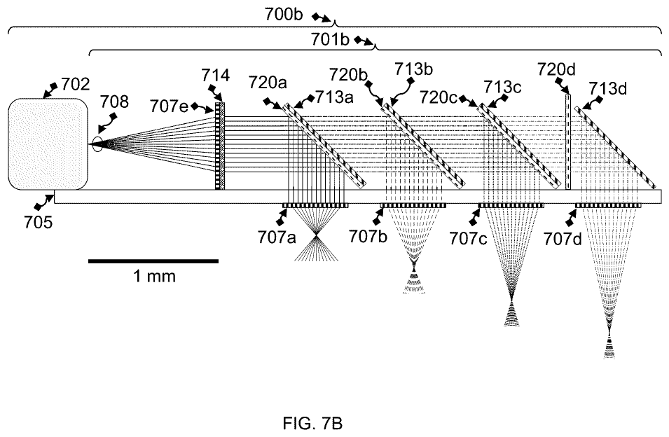

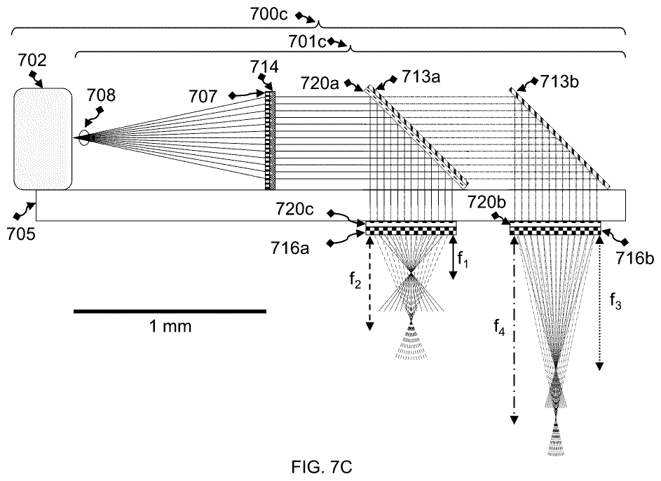

FIG. 7A-C show embodiments having Optical Imaging and Illuminating Systems with a reconfigurable focal length. In previously described embodiments, multi-zoom functionality within an OIIS has been achieved by changing the center wavelength of the input light. This may be accomplished using a tunable input light source. In FIGS. 7A-C, embodiments are described wherein the focal length of OIIS embodiment may be reconfigured utilizing Liquid Crystal (“LC”) based devices without a need to alter the wavelength of the optical source.

In the catheter system 700a shown in FIG. 7A, OIIS 701a is designed at a first wavelength (e.g., a center wavelength 800 nm). Rays 708 coupled out of fiber 702 are collimated by the diffractive lens 707d. An absorptive polarizer 714 may be stacked adjacent the diffractive lens 707d to linearly polarize the rays 708 (e.g., to have a P₁ polarization). By adjusting the input polarization, the functionality of LCGs 719a, 719b may be controlled. In some embodiments, a quarter waveplate or other type of waveplate (not shown) may be included after absorptive polarizer 714 to generate a different polarization (e.g., P₂ polarization) as desired. Polarized rays are reflected by mirror 709 toward the substrate 705. The substrate may have an anti-reflection coating on at least a first surface 705a to reduce the reflection loss when entering the substrate 705. The rays 708 are diffracted (e.g., at a diffraction angle larger than the TIR angle of the substrate) by the diffractive grating 706a toward the first Liquid Crystal Grating (“LCG”) 719a. The function of each LCG can be independently controlled by one or more electric signals (not shown). The electrical signals may be controlled by a control module and may be manually or automatically controlled. In OFF state, LCG may function as grating tuned for the wavelength of rays 708 so that the rays 708 are diffracted by the LCG 719a. In the ON state, the LCG 719a does not interact with impinging rays and the rays 708 continue in TIR through the substrate 705.

Initially, the system 700a is described having the LCG 719a in an OFF state. Rays 708 are diffracted by diffraction grating 706a toward the OFF LCG 719a. The OFF LCG 719a diffracts the rays 708 toward diffractive lens 707a where they are focused at a first focal length (e.g., a focal length of 0.5 mm). This is the end of the light path when LCG 719a is off.

In a second scenario wherein the LCG 719a is ON, rays 708 are diffracted by diffractive grating 706a toward the LCG 719a and do not interact with LCG 719a. Instead, the rays 708 are reflected by the top surface of the substrate 705 due to TIR. After reflecting from the top surface 705a, the rays 708 reach a second LCG 719b. When the second LCG 719b is OFF, rays 708 are diffracted toward diffractive lens 707b where they are focused at a second focal length (e.g., a focal length of 1.5 mm). This is the end of the light path when LCG 719a is on and LCG 719b is off.

In a third scenario, both LCG 719a and LCG 719b are ON; thus, the rays 708 will not interact with either of the first and second LCGs 719a, 719b. Rays 708 propagate through the substrate 705 in TIR until they reach the second diffractive grating 706b. After being diffracted by the grating 706b, rays 708 are focused by diffractive lens 707c at a third focal length (e.g., a focal length of 3 mm).

By switching LCGs ON and OFF, the OIIS 701a can be reconfigured such that light is emitted (and may also be collected via reciprocity of the system) at a desired focal length. In some embodiments, three discrete focal lengths of (e.g., 0.5 mm, 1 mm, and 3 mm) can be achieved. One of skill in the art will appreciate that the number of achievable focal lengths can be increased or decreased by cascading more or fewer LCGs, respectively, along with other appropriate components (e.g., diffractive gratings and/or diffractive lenses designed to have selected focal lengths).

FIG. 7B shows an embodiment wherein catheter system 700b includes a Liquid Crystal Half-Waveplate (“LCHWP”) within the OIIS 701b. The LCHWP is used to achieve a reconfigurable multi-zoom OIIS 701b. In system 700b, light rays 708 have a spectral channel with a center wavelength (e.g., 1100 nm). After exiting the fiber 702 the rays 708 are collimated by diffractive lens 707e, which is designed for the wavelength of rays 708. The collimated rays are linearly polarized (e.g., with P₁ polarization) by the absorptive polarizer 714. Polarized rays 708 interact with a first LCHWP 720a.

Initially, the light path will be described with respect to a first scenario (illustrated with solid lines) wherein the first LCHWP 720a is in the OFF state. When in the OFF state, the LCHWP 720a acts as a Half Waveplate (HWP) and changes the incoming linearly polarized rays into their orthogonal state (e.g., P₂ polarization). The rays interact with a first Polarization-Selective Reflector (“PSR”) 713a. All PSRs in the system (e.g., 713a, 713b, 713c, 713d) are co-polarized with the absorptive polarizer 714, meaning that if the linearly polarized rays 708 pass through 714, the rays 708 will also pass through the PSRs. In this example, the absorptive polarizer 714 transmits rays with P₁ polarization; PSRs transmit P₁ polarized rays and reflect P₂ polarized rays. Because rays 708 passing through the first LCHWP 720a in the OFF state switch polarization (e.g., switch from P₁ to P₂ polarization), the rays will be reflected by PSR 713a toward a diffractive lens 707a disposed on the substrate 705. The lens 707a focuses the rays 708 at a first focal length (e.g., a focal length of 0.25 mm).

In a second scenario, the first LCHWP 720a is in ON state and the second LCHWP 720b is in OFF state. In this case, after passing through 720a rays 708 do not change polarization (e.g., remain at P₁ polarization) and pass through PSR 713a after which they reach second LCHWP 720b. In the OFF state, LCHWP 720b acts as a HWP that switches the polarization of rays (e.g., from P₁ to P₂ polarization). Therefore, rays 708 passing through OFF LCHWP 720b are reflected by PSR 713b toward the diffractive lens 707b where they are focused at a second focal length (e.g., a focal length of 0.5 mm). Similarly, by turning ON first and second LCHWPs 720a, 720b and turning the LCHWP 720c OFF, rays are re-directed toward diffractive lens 707c and are focused at a third focal length (e.g., a focal length of 0.75 mm).

The last scenario is when the first three LCHWPs 720a-c are in the ON state and the fourth LCHWP 720d is in the OFF state. Polarization of rays 708 is switched by the fourth LCHWP 720d and rays 708 are reflected by PSR 713d toward diffractive lens 707d where they are focused at a fourth focal length (e.g., a focal length of 1 mm). Thus, by turning ON and OFF selected LCHWP, rays 708 may be directed to a particular diffractive lens thereby focusing light at a selected focal length. While the OIIS 701b is capable of focusing light at four discrete values (e.g., focal lengths of 0.25 mm, 0.5 mm, 0.75 mm, and 1 mm), more or fewer focal lengths may be achieved by adding or removing one or more LCHWP, PSR, and diffractive lenses.

Fourth LCHWP 720d is shown disposed at an angle (e.g., approximately 45 degrees) relative to the fourth PSR 713d while first, second, and third LCHWPs 720a-c are disposed at an angle equal to that of the first, second, and third PSRs 713a, 713b, and 713c, respectively. The angle of LCHWPs relative to the PSR can be adjusted while achieving similar results depending on the design of LCHWP. In an alternative embodiment, the fourth LCHWP 720d may be removed and PSR 713d may be reoriented in such a way that the PSR 713d (e.g., the final PSR in the series) is cross-polarized relative to absorptive polarizer 714. The resulting OIIS has similar functionality to that of OIIS 701b, but with one fewer component. Similar to other embodiments disclosed herein, this configuration may be combined with other embodiments described herein to add more functionalities such as polarization resolved imaging or multispectral imaging.

Referring now to FIG. 7C, an embodiment is shown wherein an OIIS with four reconfigurable focal lengths can be achieved. Catheter system 700c includes OIIS 701c. A ray-tracing simulation is illustrated wherein rays 708 having a first wavelength (e.g., a wavelength of 1300 nm) propagate through the system 700c. These rays are collimated by a diffractive lens 707. To ensure the rays 708 are linearly polarized at a first polarization (e.g., P₁ polarization), an absorptive polarizer 714 is placed after diffractive lens 707. In the system 700c, PSR 713a is oriented in a cross-polarized position relative to absorptive polarizer 714. Notably, the PSR 713a may be designed to be co-polarized with polarizer 714 and the resulting OIIS performs similarly to OIIS 701c. Thus, similar system functionality may be achieved by altering the orientations of components or by making other small adjustments in the design; such changes and adjustments may be matters of design choice and do not depart from the scope of the present disclosure.

Referring to FIG. 7C, a first light path is described and is illustrated with solid lines. In this first scenario, LCHWP 720a is in the ON state so that it does not change the polarization of rays (e.g., rays 708 remain at P₁ polarization). The first PSR 713a is cross-polarized with polarizer 714; therefore, the rays 708 are reflected by PSR 713a toward the LCHWP 720c on substrate 705. The LCHWP 720c is also in the ON state so it does not change the polarization of rays 708. As a result, rays 708 will keep their original polarization state (e.g., P₁ polarization) and will be focused by Polarization Selective Diffractive Lens (“PSDL”) 716a at a first focal length (e.g., f₁=0.25 mm).

If the state of LCHWP 720a remains ON and LCHWP 720c is turned OFF, the polarization of the rays 708 will be switched to the orthogonal state (e.g., P₂ polarization) by the LCHWP 720c before interacting with PSDL 716a. Because PSDL focuses light differently depending on polarization of the light, PSDL 716a will focus the P₂ polarized rays 708 at a second focal length (e.g., f₂=0.5 mm) that is different from the first focal length. PSDL 716a is designed in such a way that it focuses P₁ polarization light at focal distance f₁ and P₂ polarization light at focal distance f₂. P₁ and P₂ are two arbitrary chosen orthogonal states of linear polarization, but they may alternatively be circular or elliptical polarization while achieving an OIIS with the same functionality as OIIS 701c.

In a second scenario, LCHWP 720a is turned OFF, thereby causing the polarization of incoming rays 708 to switch into the orthogonal state (e.g., P₂ polarization). P₂ polarized rays 708 will pass through the PSR 713a and reach PSR 713b. The PSR 713b is oriented cross-polarized relative to PSR 713a; therefore, PSR 713b reflects the P₂ polarized rays toward LCHWP 720b. When the LCHWP 720b is ON, it does not change the polarization of rays 708 (e.g., rays 708 maintain P₂ polarization). The PSDL 716b will focus these rays at a third focal length (e.g., f₃=0.75 mm). However, when LCHWP 720b is turned OFF, the LCHWP 720b will switch the polarization of impinging rays. Therefore, when LCHWP 720b is OFF, rays 708 switch to P₁ polarization and PSDL 716b will focus the P₁ polarized rays 708 at a fourth focal length (e.g., f₄=1 mm) that is different from the third focal length. Thus, by appropriately changing the ON/OFF status of each LCHWP in the system and by utilizing polarization-selective diffractive lenses whose focal length is dependent on the polarization of incoming rays, an OIIS is achieved which has reconfigurable focal length using one or more input electric signals to the LCHWP components.

An OIIS with reconfigurable focal length is advantageous for depth imaging. In particular, an adjustable focal length can be used to obtain the best imaging quality at the depth of interest. In the case of illumination, the focal length may be selected to achieve the maximum intensity of light at a certain depth of tissue for therapeutic purposes or any other applications, such as tissue ablation or other laser surgical applications.

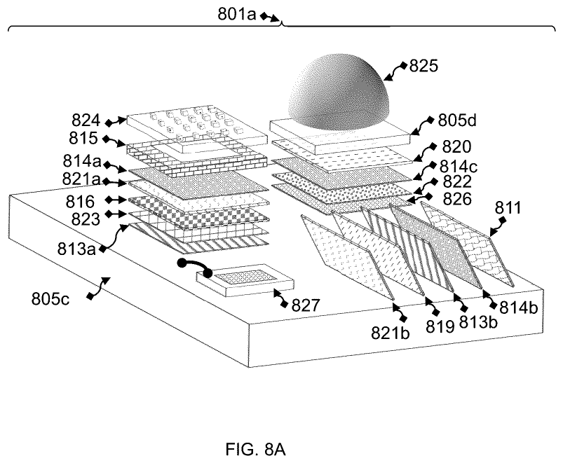

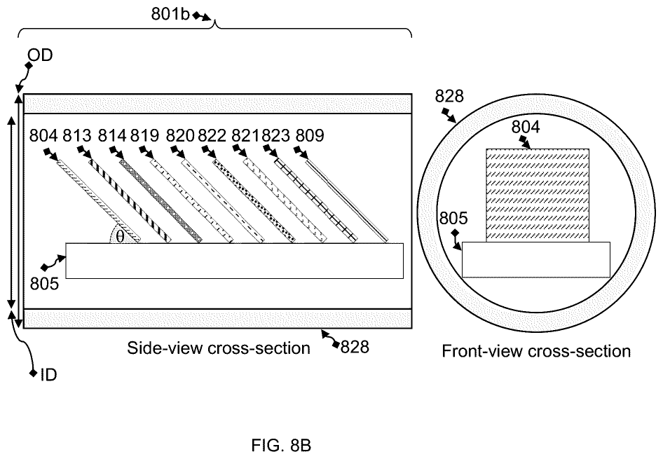

FIG. 8A illustrates an exploded view of an embodiment of an OIIS 801a illustrating different integration schemes with various components. In particular, FIG. 8A shows how one can utilize horizontal cascading and/or vertical stacking of various components to add extra functionality to the optical imaging and illuminating system. Most of the components used in the previously described embodiments have a planar form which can be easily integrated/stacked with other planar components such as substrates 805c and 805d, WSG 811, PSR 813a-b, absorptive polarizer 814a-c, spacer 815, PSDL 816, LCG 819, LCHWP 820, waveplate (“WP”) 821a-b (e.g., half-waveplate and quarter-waveplates), color filter 822, thin-film 823 (e.g., AR coating), diffractive element 824 (e.g., holograms, diffusers, sub-wavelength gratings), and angle-selective surface 826. Also, these components can be integrated or otherwise combined with refractive components such as lenses 825. This vertical integration capability can advantageously expand the functionality of the OIISs described herein. For example, by stacking liquid crystals, polarizers, and waveplates one may control/change the polarization of light as desired and/or may remove unwanted polarization. Some other examples are stacking thin-film 823 and a color filter 822 to control the reflection or transmission of light depending on its wavelength. Thin films may also be used to form an AR coating on the substrate surface or on various other components, such as fiber facets, to avoid reflection loss.

Integration of a sensor/detector 827 on the OIIS platform, which may receive feedback from the imaging/illuminating scene, is also contemplated herein. One example of a sensor is a depth sensor to measure the distance of the object to be imaged (e.g., an organ or tissue) from an OIIS to accordingly adjust its focal length or any other parameters. The adjustments may be made manually based on readings from the depth sensor or may be controlled automatically by a control module (not shown). Various electrical traces to one or more components in the OIIS, such as sensors, or electrically actuated LC-based components, may be included on substrates or other components within the OIIS. These traces are omitted from illustrations for clarity.

The components included in OIIS 801a or 801b may have an arbitrary angle with the substrate 805 as shown in FIG. 8B. For example, one or more of the components may have an angle θ with the substrate. The angle θ may be 30°, 35°, 45°, 50°, 55°, or an arbitrary value. FIG. 8B also shows an example of OIIS 801b which is enclosed by a tube 828. This tube can have an arbitrary inner diameter (ID) and outer diameter (OD). It can be also made of glass, plastic, polymer, or any other appropriate materials. This tube will be in the optical path (e.g., between the lenses of the various OIIS systems and the tissue or object to be imaged) and the lens will focus the light through this tube. Thus, the tube's contours and material may be considered in designing lenses or other components in OIIS.

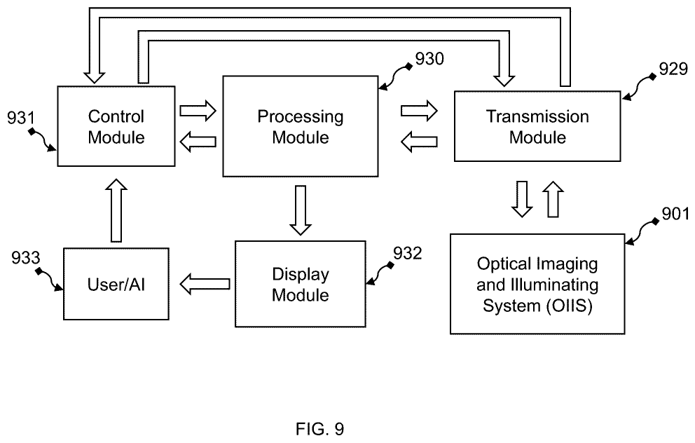

In FIG. 9, a block diagram illustrates different modules for implementing the methods disclosed herein, in accordance with some embodiments. FIG. 9 shows a high-level schematic of different modules and systems, some of which may be optional, and how they may work together to improve the performance of the whole imaging and illuminating system. The imaging and illuminating systems may include one or more OIIS embodiments as discussed previously herein. In the system shown in FIG. 9, the OIIS 901 is designed to focus light into the object and/or collect scatter light from the object to form an image. The OIIS 901 receives the input light from the Processing Module 930 via the Transmission Module 929. The Transmission Module may include one or more single-mode fibers, one or more photonic-crystal fibers, and/or one or more multimode fibers. Fibers transmit the input light from a source (e.g., laser, LED, supercontinuum, swept-source) to the OIIS and then collect the image information from the OIIS for transmission back to the Processing Module 930. Furthermore, the Transmission Module 929 may include at least one electrical wire and/or at least one wireless transmitter. Electrical wire and/or the wireless transmitter can be used to transmit an electrical or electromagnetic signal between the sensor (see sensor/detector 827 in FIG. 8A) and the Processing Module. The Processing Module may include at least one interferometric arm (in the case of optical coherence tomography imaging) for image processing purposes, at least one photodetector, at least one camera, at least one imaging sensor, at least one fiber coupler (e.g., 50/50 fiber coupler, 30/70 fiber coupler, 20/80 fiber coupler, 10/90 fiber coupler), and/or at least one spectrometer. All of these components in the Processing Module may be collectively used to form and analyze images and send them to the Display Module 932. User/Artificial Intelligence (“AI”) Module 933 receives image information from the Display Module and then decides which parameters in the Processing Module or OIIS need to be changed/adjusted to improve the image quality. A User and/or an AI 933 analyzes the data and makes required changes and adjustments via the Control Module 931, Processing Module 930, and Transmission Module 929.

The foregoing description and figures are illustrative of various embodiments of the present invention and are not to be construed as limiting thereof. Although a few exemplary embodiments of this invention have been specifically described, those skilled in the art will readily appreciate that many modifications are possible in the exemplary embodiments without materially departing from the novel teachings and advantages of this disclosure. Accordingly, many different embodiments stem from the above description and the drawings.

1. An optical system for an endoscope comprising:
a substrate having a first surface and a second surface, wherein at least one of the surfaces is oriented substantially parallel to an axial direction of a fiber configured to deliver light propagating along the axial direction; and
an optical component supported by the substrate, the optical component directing the light from the fiber into at least two light paths, wherein each of the two light paths is re-directed to a transverse direction and focused to a different focus spot located to a side of the optical system.

2. The optical system of claim 1, wherein the optical component comprises an optical reflector, light exits the fiber propagating along the axial direction and is directed by the optical reflector towards the substrate and propagates through the substrate to the corresponding focus spot.

3. The optical system of claim 1, wherein the optical component is mounted on the first surface and extends in a transverse direction away from the first surface.

4. The optical system of claim 3, wherein the optical component comprises a wavelength-selective reflector mounted at an angle relative to the first surface.

5. The optical system of claim 1, further comprising:
a diffractive lens positioned flat on one of the surfaces, wherein one of the light paths exits the substrate in a transverse direction and the diffractive lens focuses that light path to the corresponding focus spot.

6. The optical system of claim 1, further comprising:
an axicon positioned flat on one of the surfaces, wherein one of the light paths exits the substrate in a transverse direction and the axicon focuses that light path to the corresponding focus spot with an extended depth of focus.

7. The optical system of claim 1, wherein the different focus spots have different focal lengths.

8. The optical system of claim 1, wherein the different focus spots have different depths of focus.

9. The optical system of claim 1, wherein the two light paths comprise two different spectral channels.

10. The optical system of claim 1, wherein the two light paths comprise two different polarization channels.

11. The optical system of claim 1, further comprising:
a set of at least two optical components supported by the substrate, the set of optical components directing the light from the fiber into at least three light paths, wherein the three light paths comprise at least two of different focus parameters, different wavelengths and different polarizations.

12. An endoscopic catheter comprising:
an optical fiber having two ends;
a fiber connector connected to one end of the optical fiber; and
an optical system connected to an opposite end of the optical fiber; wherein the optical system comprises:
a substrate having a first surface and a second surface, wherein at least one of the surfaces is oriented substantially parallel to an axial direction of the optical fiber and the optical fiber delivers light to the optical system propagating along the axial direction; and
an optical component supported by the substrate, the optical component directing the light from the optical fiber into at least two light paths, wherein each of the two light paths is re-directed to a transverse direction and focused to a different focus spot located to a side of the optical system.

13. The endoscopic catheter of claim 12, further comprising:
a ferrule that connects the optical system to the optical fiber; and
a torque coil that rotates the optical system.

14. The endoscopic catheter of claim 12, wherein the optical system has a cross-section of not more than 1.5 mm×1.5 mm and a length of not more than 5 mm.

15. The endoscopic catheter of claim 12, wherein the optical system also collects light scattered from tissue located at the focus spots via propagation along a reverse direction through the two light paths.

16. The endoscopic catheter of claim 12, further comprising:
a controller, wherein the optical component is wavelength-sensitive or wavelength-selective and the controller adjusts a wavelength composition of the light delivered by the fiber.

17. The endoscopic catheter of claim 12, further comprising:
a controller, wherein the optical component is wavelength-sensitive or wavelength-selective and the controller adjusts a wavelength sensitivity or wavelength selectivity of the optical component.

18. The endoscopic catheter of claim 12, further comprising:
a controller, wherein the light paths contain at least one electro-optic component and the controller adjusts the electro-optic component.

19. The endoscopic catheter of claim 12, wherein the two light paths comprise two different spectral channels.

20. The endoscopic catheter of claim 12, wherein the two light paths comprise a diffractive lens that focuses two different polarization channels to two different focal lengths.
